SEQUENCE LISTING (23 sequences)

SEQ ID NO: 1 - protein, 192 aa, Artificial Sequence
  synthetic
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEN

SEQ ID NO: 2 - protein, 195 aa, Artificial Sequence
  synthetic
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESA

SEQ ID NO: 3 - protein, 195 aa, Artificial Sequence
  synthetic
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTET

SEQ ID NO: 4 - protein, 195 aa, Artificial Sequence
  synthetic
PEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSK

SEQ ID NO: 5 - protein, 40 aa, Artificial Sequence
  synthetic
RIARLEEKVKTLKAQNSELASTANMLREQVAQLKQKVMNH

SEQ ID NO: 6 - protein, 40 aa, Artificial Sequence
  synthetic
LTDTLQAETDQLEDEKSALQTEIANLLKEKEKLEFILAAH

SEQ ID NO: 7 - protein, 15 aa, Artificial Sequence
  synthetic
PVSKMRMATPLLMQA

SEQ ID NO: 8 - protein, 10 aa, Artificial Sequence
  synthetic
EQKLISEEDL

SEQ ID NO: 9 - protein, 9 aa, Artificial Sequence
  synthetic
YPYDVPDYA

SEQ ID NO: 10 - protein, 314 aa, Artificial Sequence
  synthetic
MKVLIVLLAIFAALPLALAQPVISTTVGSAAEGSLDKREARPIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENGAGGGGSLEVLFQGPGGGRIARLEEKVKTLKAQNSELASTANMLREQVAQLKQKVMNHVDGGHHHHHHPWEQKLISEEDL

SEQ ID NO: 11 - protein, 404 aa, Artificial Sequence
  synthetic
MKVLIVLLAIFAALPLALAQPVISTTVGSAAEGSLDKREARGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESALKGGGGSLEVLFQGPGGGLTDTLQAETDQLEDEKSALQTEIANLLKEKEKLEFILAAHTSGGDYKDDDDKGGGGSGGGGSQELTTICEQIPSPTLESTPYSLSTTTILANGKAMQGVFEYYKSVTFVSNCGSHPSTTSKGSPINTQYVFKDNSSTIEGRYPYDVPDYA

SEQ ID NO: 12 - protein, 402 aa, Artificial Sequence
  synthetic
MKVLIVLLAIFAALPLALAQPVISTTVGSAAEGSLDKREAREDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETGGGGSLEVLFQGPGGGLTDTLQAETDQLEDEKSALQTEIANLLKEKEKLEFILAAHTSGGDYKDDDDKGGGGSGGGGSQELTTICEQIPSPTLESTPYSLSTTTILANGKAMQGVFEYYKSVTFVSNCGSHPSTTSKGSPINTQYVFKDNSSTIEGRYPYDVPDYA

SEQ ID NO: 13 - protein, 317 aa, Artificial Sequence
  synthetic
MKVLIVLLAIFAALPLALAQPVISTTVGSAAEGSLDKREARPPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKGTGGGGSLEVLFQGPGGGRIARLEEKVKTLKAQNSELASTANMLREQVAQLKQKVMNHVDGGHHHHHHPWEQKLISEEDL

SEQ ID NO: 14 - protein, 402 aa, Artificial Sequence; identical to SEQ ID NO: 12
  synthetic

SEQ ID NO: 15 - protein, 348 aa, Artificial Sequence
  synthetic
MKVLIVLLAIFAALPLALAQPVISTTVGSAAEGSLDKREARPPVSKMRMATPLLMQAGGGGSLVPRGSGGGGSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKGTGGGGSLEVLFQGPGGGRIARLEEKVKTLKAQNSELASTANMLREQVAQLKQKVMNHVDGGHHHHHHPWEQKLISEEDL

SEQ ID NO: 16 - protein, 79 aa, Artificial Sequence
  synthetic
QELTTICEQIPSPTLESTPYSLSTTTILANGKAMQGVFEYYKSVTFVSNCGSHPSTTSKGSPINTQYVFKDNSSTIEGR

SEQ ID NO: 17 - protein, 41 aa, Artificial Sequence
  synthetic
KVLIVLLAIFAALPLALAQPVISTTVGSAAEGSLDKREARP

SEQ ID NO: 18 - protein, 18 aa, Artificial Sequence
  synthetic
GAGGGGSLEVLFQGPGGG

SEQ ID NO: 19 - protein, 12 aa, Artificial Sequence
  synthetic
VDGGHHHHHHPW

SEQ ID NO: 20 - protein, 18 aa, Artificial Sequence
  synthetic
LKGGGGSLEVLFQGPGGG

SEQ ID NO: 21 - protein, 22 aa, Artificial Sequence
  synthetic
TSGGDYKDDDDKGGGGSGGGGS

SEQ ID NO: 22 - protein, 16 aa, Artificial Sequence
  synthetic
GGGGSLEVLFQGPGGG

SEQ ID NO: 23 - protein, 18 aa, Artificial Sequence
  synthetic
GTGGGGSLEVLFQGPGGG

(19) United States
(12) Patent Application Publication
(10) Pub. No.: US 2022/0381793 A1
(43) Pub. Date: Dec. 1, 2022

(54) COMPOSITIONS AND METHODS FOR IDENTIFYING MHC-II BINDING PEPTIDES

(71) Applicant: The Board of Trustees of the Leland Stanford Junior University, Stanford, CA (US)

(72) Inventors: Wei JIANG, Palo Alto, CA (US); Elizabeth D. MELLINS, Palo Alto, CA (US)

(73) Assignee: The Board of Trustees of the Leland Stanford Junior University, Stanford, CA (US)

(21) Appl. No.: 17/824,980

(22) Filed: May 26, 2022

Related U.S. Application Data
(60) Provisional application No. 63/194,741, filed on May 28, 2021.

(51) Int. Cl. G01N 33/68; C12N 1/16; C12N 15/62; C12N 15/81

(52) U.S. Cl. CPC G01N 33/6878; C12N 1/16; C12N 15/625; C12N 15/815; C12N 2800/102; G01N 2333/70539

(57) ABSTRACT

Provided herein, inter alia, are methods and compositions for identifying major histocompatibility class II (MEW II) complex binding peptides.

### CROSS REFERENCE TO RELATED APPLICATIONS

The present application claims the benefit of priority under 35 U.S.C. § 119 to U.S. Provisional Application Ser. No. 63/194,741, titled “COMPOSITIONS AND METHODS FOR IDENTIFYING MHC-II BINDING PEPTIDES” and filed May 28, 2021, the disclosure of which is incorporated herein by reference in its entirety.

### INCORPORATION BY REFERENCE OF SEQUENCE LISTING

The present application contains a sequence listing which has been submitted in ASCII format via EFS-Web. The content of the computer readable ASCII text file named “6045.0574_ST25.txt”, which was created on Jul. 27, 2022 and is 31 KB in size.

### BACKGROUND

CD4+ T cells respond to antigen via the interaction between the T cell receptor (TCR) and antigen-derived peptides bound by heterodimeric (α and β) major histocompatibility complex class II (MHC-II) molecules on the surface of professional antigen-presenting cells (APCs). Using characterized peptide/MHC-II as probes for T cell targeting and TCR identification is essential in research and therapeutic inventions that are based on T-dependent immunity. Peptide/MHC-II characterization requires the screening of overlapping peptides from a particular protein antigen for identification of peptide ligands that bind a particular MHC-II allele. Most current methods express single MHC-II allele in model cell lines and require purification of the expressed MHC-II protein for peptide elution (a mass-spectrometry-based method) or peptide binding studies. In humans, MHC-II contain human leucocyte antigen (HLA)-DR, -DQ, and -DP sub-types, each with hundreds of alleles yielding thousands of allelic protein variants. Thus, creating mammalian or insect cell lines to express individual MHC-II alleles and purifying them one by one for peptide/MHC-II characterization is both labor-intensive and cost-inefficient.

Disclosed herein, inter alia, are solutions to these and other problems in the art.

### BRIEF SUMMARY OF THE INVENTION

In an aspect is provided a cell including a major histocompatibility class II (MHC II) complex, wherein the MHC II complex includes an alpha chain and a beta chain, wherein the alpha chain is attached to a first protein binding domain and the beta chain is attached to a second protein binding domain, wherein the first protein binding domain is bound to the second protein binding domain to form a MHC II complex.

A nucleic acid encoding a major histocompatibility class II (MHC II) complex, wherein the MHC II complex includes an alpha chain and a beta chain, wherein the alpha chain is attached to a first protein binding domain and the beta chain is attached to a second protein binding domain, wherein the first protein binding domain and the second protein binding domain are capable of non-covalently binding to form a MHC complex

A method of making a major histocompatibility class II (MHC II) complex, the method including transforming a cell with a nucleic acid provided herein including embodiments thereof, and culturing the cell under conditions wherein the MHC II complex is expressed.

A method of identifying a peptide that binds a major histocompatibility class II (MHC II) complex, the method including: i) contacting a cell provided herein including embodiments thereof with a peptide, and detecting binding of the peptide to the MHC II complex, thereby identifying the MHC II complex binding peptide.

### BRIEF DESCRIPTION OF THE DRAWINGS

FIGS. 1A-1D. Leucine zipper enhanced surface expression of DR4 in yeast. FIG. 1A. Gene construct used for yeast display of the non-covalent DR4 ectodomain with or without covalently linked HA₃₀₆₋₃₁₈, a hemagglutinin-derived peptide. The bi-directional GAL1-10 promoter directs the expression of α and β chains, respectively. Fos/Jun LZ motifs in the last two constructs are used to facilitate α/β pairing. FIG. 1B. Schematic illustration of appropriate assembled HA₃₀₆₋₃₁₈/DR4 or “empty” DR4, as a fusion to the yeast surface protein agglutinin, composed of the Aga1p and Aga2p subunits. Arrows indicate protein or epitope tags for antibody staining and detection by flow cytometry. FIG. 1C. Expression of HA₃₀₆₋₃₁₈/DR4 and “empty” DR4 on the surface of yeast analyzed by flow cytometry. Yeast cells transformed with one of the constructs shown in FIG. 1A were induced for protein expression and double-stained with anti-DRαβ (clone L243) and anti-HA-tag antibodies. Background staining uses untransformed yeast (EBY100). FIG. 1D. Comparison of DR4 expression levels in different yeast transformants. Fold change of median fluorescence intensity (MFI) of DRαβ or HA-tag signal on the surface of transformants over background (BG) is quantified. Representative histograms are shown to the right. Error bars represent standard error of the mean (SEM) from at least three independent experiments. The significance was determined using one-way ANOVA test. ns: p>0.05, **: p<0.01, ***: p<0.001, ****: p<0.0001.

FIGS. 2A-2D. Yeast surface display of peptide-linked or “empty” DQ6 with LZ. FIG. 2A. Gene construct used for expression of the noncovalent DQ6 ectodomain with or without specific peptides covalently linked to the DQ6β N terminus. In the peptide-linked constructs, we included two peptides, HCRT₈₇₋₉₇ and HA₂₇₃₋₂₈₆, who's binding to DQ6 has been well characterized, and a positive control Ii peptide CLIP₈₇₋₁₀₁. Unlike the DR construct, the a chain followed by the Fos LZ motif is at the upstream of AGA2. FIG. 2B. Schematic illustration of appropriate assembled peptide-linked or “empty” DQ6, as a fusion to the yeast surface protein agglutinin, similar to FIG. 1B. FIG. 2C. Expression of peptide/DQ6 or “empty” DQ6 on the surface of yeast analyzed by flow cytometry, as in FIG. 1c, except that L243 was replaced by an anti-DQαβ antibody (clone SPV-L3). FIG. 2D. Comparison of DQ6 expression levels in different yeast transformants. Fold change of MFI over BG is quantified as in FIG. 1D. Representative histograms are shown to the right. Error bars represent SEM from at least three independent experiments. The significance was determined using one-way ANOVA test. ns: p>0.05, **: p<0.01, ***: p<0.001.

FIGS. 3A-3E. Binding of peptides to “empty” MHC-II on yeast. FIG. 3A. Yeast expressing “empty” MHC-II was incubated with 20 μM biotinylated peptides (Bio-HA₃₀₆₋₃₁₈ for DR4 or HCRT₁₋₁₃-Bio for DQ6) in different pH buffers at the indicated temperature for 20 hours, followed by streptavidin (SA)-Alexa fluor 647 staining and flow cytometric analysis. Fold change of MFI_{SA} over negative (an irrelevant MHCIa peptide) is quantified. FIG. 3B. Representative flow cytometric measurement for MFI_{SA} of biotinylated peptides bound by MHC-II. Incubation of yeast and peptides was performed at pH 5.0, 30° C. for 20 hours. The right panel contains 200 μM non-biotinylated ligands (HA₃₀₆₋₃₁₈ for DR4 or HCRT₈₅₋₉₉ for DQ6) as competitors. FIG. 3C. Yeast was incubated with 20 μM biotinylated peptides at pH 5.0, 30° C. for various time intervals and analyzed. The binding signals normalized with negative control were plotted against time and fitted to calculate observed association rate constant (Kobs). FIG. 3D. Yeast was incubated with different concentrations of biotinylated peptides at pH 5.0, 30° C. for 20 hours to reach equilibrium. Data were fitted to calculate apparent equilibrium dissociation constant (KDapp). FIG. 3E. Yeast was incubated with 20 μM biotinylated peptides and various concentrations of competitor peptides (HA₃₀₆₋₃₁₈ for DR4 or HCRT₈₅₋₉₉ for DQ6) at pH 5.0, 30° C. for 20 hours and analyzed. % binding was determined by (MFI_{with competitor}−BG)/(MFI_{no competitor}−BG)×100% and fitted to calculate IC50. All error bars represent SEM from three independent experiments.

FIGS. 4A-4C. RIPPA identified all DQ6 binders from HCRT. FIG. 4A. Yeast expressing “empty” DQ6 was incubated with 20 μM HCRT₁₋₁₃-Bio and 200 μM of the indicated non-biotinylated HCRT 15-mer peptide at pH 5.0, 30° C. for 20 hours and analyzed by flow cytometry. % Competition=100%−% binding (% binding as calculated in FIG. 3E). Error bars represent SEM from three independent experiments. Bolded letters denote previously identified 9-aa DQ6-binding registers^{4, 5, 42}: LPSTTKVSWA, SSGAAAQPL, NHAAGILTL and NHAAGILTM, respectively. HCRT₄₉₋₆₃ or HCRT₈₁₋₉₅ covers <8 aa of the previously determined registers. Binding ranks for each HCRT peptide predicted by NetMHCIIpan-4.0 (trained on both elution and binding data) are shown as 100-% rank to the left. FIGS. 4B-4C. Correlation analysis for binding data acquired using “empty” DQ6 on yeast versus soluble CLIP/DQ6 protein (FIG. 4B) or versus NetMHCIIpan-4.0 prediction (FIG. 4C). Arrows indicate peptides that show binding in one method but not the other in the comparison. Open circles in FIG. 4B match open bars in FIG. 4A, indicating ligands identified by both empirical methods.

FIG. 5. DR4 binding peptides from the SARS-CoV-2 S protein determined by experiment versus prediction. Yeast expressing “empty” DR4 was incubated with Bio-HA₃₀₆₋₃₁₈ and 200 μM of the indicated non-biotinylated spike peptide at pH 5.0, 30° C. for 20 hours and analyzed by flow cytometry. % Competition=100%−% binding (% binding as calculated in FIG. 3E). Error bars represent SEM from three independent experiments. Binding ranks for each spike peptide predicted by NetMHCIIpan-4.0 were shown as 100-% rank to the left. The number in front of each peptide indicates the start position in the S precursor. Peptides that are both predicted to bind DR4 (>90%, top 10% rank) and capable of competing with Bio-HA₃₀₆₋₃₁₈ in the DR4 binding experiment are indicated in red (>75% competition, strong binders) or magenta (50-75% competition, weak binders); those that are predicted at % rank>10% but show binding in the experiment are indicated in blue (>75% competition) or cyan (50-75% competition); those that are predicted to bind DR4 but unable to compete with Bio-HA₃₀₆₋₃₁₈ in the experiment are indicated in brown. Gray area indicates peptides from the receptor-binding domain (RBD) of the S protein.

FIGS. 6A-6B. Strong DR4 binders from the SARS-CoV-2 S protein. FIG. 6A. Schematic illustration of the relative locations of RIPPA-identified peptide binders (greater or close to 75% competition in FIG. 5) to different domains of the S precursor. S with the start residue position under a bar indicates each peptide. FIG. 6B. Comparison of RIPPA-identified DR4 binders with previously identified candidate DR4-restricted T cell epitopes^{6, 13} that are either from the SARS-CoV-2 S (S in short) or SARS-CoV-1 S protein. Identical or conserved residues are bolded. The start residue position of each S or SARS-CoV-1 S peptide and the cartoon of the S peptide on the crystal structure are indicated. The structure⁴⁵ uses PDB code 6XR8 with protomer A in yellow and the other two protomers in gray. % rank_BA in FIG. 6A indicates the prediction was performed only considering binding affinity data, and the corresponding affinities are indicated in parenthesis in FIG. 6B. Individuals showing positive CD4+ T cell responses to the two SARS-CoV-2 candidate epitopes carry DR4 allelic subtypes DRB1:04:04 or 04:10 (ref⁶) as indicated in parenthesis. DRB1:04:04 and DRB1:04:04 allelic proteins have (K71R, G86V) and (D57S K71R, G86V) substitutions, respectively, which may influence peptide binding. The color scheme of peptides in both FIG. 6A and FIG. 6B is the same as in FIG. 5.

FIGS. 7A-7B. Expression of both chains of DR4 in yeast analyzed by flow cytometry. FIG. 7A. Yeast cells transformed with HA306-318/DR4-LZ or “empty” DR4-LZ constructs were induced for protein expression and double-stained with anti-HA-tag and anti-c-Myc-tag antibodies to confirm that both chains of DR4 in the LZ constructs were expressed by yeast. FIG. 7B. Comparison of DRα or β expression in the two yeast transformants as in FIG. 7A. Fold change of MFI of HA-tag or c-Myc-tag signal on the surface of transformants over BG is quantified as in FIG. 1D. Representative histograms are shown to the right. Error bars represent standard error of the mean (SEM) from at least three independent experiments. One-way ANOVA test was used for comparison. No significant difference in expression of either chain was observed between HA306-318/DR4-LZ and “empty” DR4-LZ (ns: p>0.05).

FIGS. 8A-8B. Expression of both chains of DQ6 in yeast analyzed by flow cytometry. FIG. 8A. Yeast cells transformed with peptide/DQ6-LZ or “empty” DQ6-LZ constructs were induced for protein expression and double-stained with anti-HA-tag and anti-c-Myc-tag antibodies to confirm that both chains of DQ6 in the LZ constructs were expressed by yeast. Background staining of untransformed yeast (EBY100) is shown. FIG. 8B. Comparison of DQα or β expression in the four yeast transformants as in FIG. 8A. Fold change of MFI over BG is quantified as in FIG. 7B. Representative histograms are shown to the right. Error bars represent standard error of the mean (SEM) from at least three independent experiments. The significance was determined using one-way ANOVA test. ns: p>0.05, *: p<0.05, **: p<0.01, ***: p<0.001. The expression level of DQ6 β chain, represented by fold change of c-Myctag staining over background staining, was increased significantly for CLIP87-101/DQ6 versus the other three constructs.

FIGS. 9A-9D. Representative flow cytometric histograms showing the streptavidin staining of yeast quantified in FIGS. 3A, 3C, 3D, and 3E, respectively.

FIG. 10. Representative flow cytometric histograms showing the streptavidin staining of yeast quantified in FIGS. 4A-4C.

FIGS. 11A-11B. Comparison of binding data acquired using the yeast display system versus by prediction. FIG. 11A. Correlation analysis for binding data that are shown in FIG. 5.

FIG. 11B. Competitive binding to “empty” DR4 on yeast by alternative peptides from the five regions that generate peptides predicted to bind DR4 (potential false positives) but unable to compete with Bio-HA306-318 in the experiment (peptides shown in brown in FIG. 5). Bolded letters denote amino acid residues overlapping with the corresponding false positive candidate.

### DETAILED DESCRIPTION

Provided herein, inter alia, are compositions and methods for identifying MHC II complex binding peptides. The compositions and methods allow for identification of T cell epitopes both experimentally and computationally. The methods further allow production of MHC II complex in functional formats with minimal or no modifications at the functional domains as compared to prior methods for generating the MHC II complex.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., MHC II alpha chain, MHC II alpha chain, etc.) in other proteins with different numbering systems. For example, by performing a simple sequence alignment with a protein (e.g., MHC II alpha chain, MHC II alpha chain, etc.) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970)J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990)J Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “MHC II” or “MHC II complex” as used herein refers to the major histocompatibility class II (MHC II) complex molecules typically found on antigen presenting cells, including dendritic cells, B cells and phagocytes. The MHC II complex assists in regulating the immune system, for example, by presenting antigenic peptides. An MHC II complex includes two human leukocyte antigen (HLA) proteins referred to herein as “alpha chain” or “α chain” and “beta chain” or “β chain”, which associate to form a heterodimer. The alpha1 (α1) and beta1 (β1) regions of the alpha chain and beta chain, respectively, are close in proximity and come together to form a peptide-binding domain. The alpha2 (α2) and beta2 (β2) regions of the alpha chain and beta chain, respectively, are situated closer to the cell membrane, and form an immunoglobulin-like domain. The N-terminus region of the alpha and beta chains includes the alpha1 (α1) and beta1 (β1) regions, and the C-terminus region of the alpha and beta chains includes the alpha2 (α2) and beta2 (β2) regions. The alpha chain and beta chain are typically attached to the cell surface by transmembrane domains.

The term “human leukocyte antigen” or “HLA” refers to the group of proteins encoded by the major histocompatibility complex (MHC) gene complex in humans. HLA genes encoding for the group of HLA proteins have different alleles, thus providing different functionality to the gene products. HLA proteins belonging to MHC class II (e.g. HLA-DP, HLA-DQ, HLA-DR) typically present peptides from outside of the cell. MHC class II proteins include HLA-DP, HLA-DQ, and HLA-DR, heterodimer cell-surface receptors including an alpha chain and beta chain.

The term “HLA-DR4 alpha chain” or “HLA-DR4 alpha chain protein” as provided herein includes any of the recombinant or naturally-occurring forms of the human leukocyte antigen (HLA) HLA-DR4 alpha chain protein, also known as MHC class II antigen DRA, MHC class II antigen DRA or variants or homologs thereof that maintain HLA-DR4 alpha chain protein activity (e.g. within at least 50%, 80%, 90%, 95%, 96%, 97%, 98%, 99% or 100% activity compared to HLA-DR4 alpha chain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HLA-DR4 alpha chain polypeptide. In embodiments, HLA-DR4 alpha chain is the protein as identified by the UniProt sequence reference P01903, homolog or functional fragment thereof. In embodiments, HLA-DR4 alpha chain includes the amino acid sequence of SEQ ID NO:1. In embodiments, HLA-DR4 alpha chain is the amino acid sequence of SEQ ID NO:1.

The term “HLA-DR4 beta chain” or “HLA-DR4 beta chain protein” as provided herein includes any of the recombinant or naturally-occurring forms of the human leukocyte antigen (HLA) HLA-DR4 beta chain protein, also known as MHC class II antigen DRB4, HLA-DRB4 or variants or homologs thereof that maintain HLA-DR4 beta chain protein activity (e.g. within at least 50%, 80%, 90%, 95%, 96%, 97%, 98%, 99% or 100% activity compared to HLA-DR4 beta chain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HLA-DR4 beta chain polypeptide. In embodiments, HLA-DR4 beta chain is the protein as identified by the UniProt sequence reference P13762, homolog or functional fragment thereof. In embodiments, HLA-DR4 beta chain includes the amino acid sequence of SEQ ID NO:2. In embodiments, HLA-DR4 beta chain is the amino acid sequence of SEQ ID NO:2.

The term “HLA-DQ6 alpha chain” or “HLA-DQ6 alpha chain protein” as provided herein includes any of the recombinant or naturally-occurring forms of the human leukocyte antigen (HLA) DQ alpha 1 chain (HLA-DQ6 alpha chain), also known as DC-1 alpha chain, DC-alpha, HLA-DCA, MHC class II DQA1 or variants or homologs thereof that maintain HLA-DQ6 alpha chain protein activity (e.g. within at least 50%, 80%, 90%, 95%, 96%, 97%, 98%, 99% or 100% activity compared to HLA-DQ6 alpha chain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HLA-DQ6 alpha chain polypeptide. In embodiments, HLA-DQ6 alpha chain is the protein as identified by the UniProt sequence reference P01909, homolog or functional fragment thereof. In embodiments, HLA-DQ6 alpha chain includes the amino acid sequence of SEQ ID NO:3. In embodiments, HLA-DQ6 alpha chain is the amino acid sequence of SEQ ID NO:3.

The term “HLA-DQ6 beta chain” or “HLA-DQ6 beta chain protein” as provided herein includes any of the recombinant or naturally-occurring forms of the human leukocyte antigen (HLA) DRB1 beta chain 1 protein (HLA-DQ6 beta chain), also known as MHC class II antigen DQB1. HLA-DQB1 or variants or homologs thereof that maintain HLA-DQ6 beta chain protein activity (e.g. within at least 50%, 80%, 90%, 95%, 96%, 97%, 98%, 99% or 100% activity compared to HLA-DQ6 beta chain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HLA-DQ6 beta chain polypeptide. In embodiments, HLA-DQ6 beta chain is the protein as identified by the UniProt sequence reference P01920, homolog or functional fragment thereof. In embodiments, HLA-DQ6 beta chain includes the amino acid sequence of SEQ ID NO:4. In embodiments, HLA-DQ6 beta chain is the amino acid sequence of SEQ ID NO:4.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “transforming” or “transformation” is typically used to describe introduction of a nucleic acid molecule into a bacteria or non-animal eukaryotic cell (e.g. a yeast cell), including plant cells. Transformation typically refers to DNA transfer into a cell by a non-viral method. Transformation methods usually include three main steps including: preparation of competent cells, transformation with the nucleic acid molecules (e.g. plasmid DNA), and subsequent plating to select successfully transformed cells. Common methods used in transformation of yeast cells are lithium, electroporation, biolistic and glass bead methods.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide. Any appropriate method known in the art for conjugating a peptide to the label may be employed, e.g., using methods described in Hermanson, Bioconjugate Techniques 1996, Academic Press, Inc., San Diego.

“Contacting” is used in accordance with its plain ordinary meaning and refers to the process of allowing at least two distinct species (e.g. peptide and MHC II complex) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a nucleic acid as provided herein and a cell. In embodiments contacting includes, for example, allowing a nucleic acid n as described herein to enter a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, or ability to produce progeny, etc. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, a recombinant protein is a protein is produced from a recombinant nucleic acid molecule. The nucleic acid molecule may include genetic material from multiple sources, thereby including sequences that are not otherwise naturally occurring. The recombinant DNA may be produced through methods known in the molecular biology arts or through synthetic methods. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism. For example, a protein endogenous to a yeast cell refers to a protein that is naturally expressed by the yeast cell. A nucleic acid encoding an endogenous protein may be introduced into the cell, thereby allowing expression of the protein. For example, a nucleic acid encoding Aga2p may be introduced into a yeast cell, thereby allowing expression of Aga2p.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

### Cell Compositions

Provided herein, inter alia, are cells including a major histocompatibility class II (MHC II) complex, wherein the MHC II complex includes an alpha and beta chain. The alpha chain and beta chain are attached to a first protein binding domain and a second protein binding domain, respectively. The first and second protein binding domains bind, thereby allowing formation of the MHC II complex. In instances, one of the alpha chain or beta chain is attached to a protein expressed on the surface of the cell, thereby allowing attachment of the MHC II complex to the cell surface. For example, the alpha chain, first protein binding domain and cell surface protein may form a fusion protein, thereby anchoring the alpha chain to the cell.

Thus, in an aspect is is provided a cell including a major histocompatibility class II (MHC II) complex, wherein the MHC II complex includes an alpha chain and a beta chain, wherein the alpha chain is attached to a first protein binding domain and the beta chain is attached to a second protein binding domain, wherein the first protein binding domain is bound to the second protein binding domain to form a MHC II complex. In embodiments, the cell is a yeast cell. In embodiments, the MHC II complex is bound to the surface of the cell through attachment of the alpha chain or the beta chain to a molecule on the cell surface. In embodiments, the MHC II complex is bound to the surface of the cell through attachment of the alpha chain to the molecule on the cell surface. In embodiments, the MHC II complex is bound to the surface of the cell through attachment of the beta chain to the molecule on the cell surface. In embodiments, the alpha chain is covalently attached to the molecule on the cell surface. In embodiments, the beta chain is covalently attached to the molecule on the cell surface.

In embodiments, the molecule is a protein. In embodiments, the protein is endogenous to the cell. In embodiments, the protein is Aga2p, a-agglutinin, a-agglutinin, flocculin, Cwp1p, Cwp2p or Tip1p. In embodiments, the protein is a-agglutinin. In embodiments, the protein is a-agglutinin. In embodiments, the protein is flocculin. In embodiments, the protein is Cwp1p. In embodiments, the protein is Cwp2p. In embodiments, the protein is Tip1p. In embodiments, the protein is Aga2p. In embodiments, Aga2p includes the amino acid sequence of SEQ ID NO:16. In embodiments, Aga2p is the amino acid sequence of SEQ ID NO:16.

The protein may be any protein expressed on the surface of the cell, thereby allowing binding of the MHC II complex to the cell surface. Proteins and methods that may be used to bind the MHC II complex to the surface of the cell are described, for example, in Pepper. L. R. et al., A decade of yeast surface display technology: Where are we now? Comb Chem High Throughput Screen. 2008 February; 11(2): 127-134, which is incorporated herein in its entirety and for all purposes. In embodiments, the protein is part of a fusion protein including the first protein binding domain and the alpha chain. In embodiments, the protein is part of a fusion protein including the second protein binding domain and the beta chain.

In embodiments, the first protein binding domain is non-covalently bound to the second protein binding domain. In embodiments, the first protein binding domain is covalently bound to said second protein binding domain. In embodiments, the first protein binding domain is a first leucine zipper domain and the second protein binding domain is a second leucine zipper domain. In embodiments, the first leucine zipper domain includes the sequence of SEQ ID NO:5 and the second leucine zipper domain includes the sequence of SEQ ID NO:6. In embodiments, the first leucine zipper domain includes the sequence of SEQ ID NO:6 and the second leucine zipper domain includes the sequence of SEQ ID NO:5.

As used herein, “leucine zipper domain” refers to a protein structural motif that includes an alpha helical structure. The alpha helix includes a periodic leucine residue at approximately every seventh position over approximately eight helical turns of the structure. The leucine side chains from a first leucine zipper domain is capable of interdigitating with leucine side chains from a second leucine zipper domain, thereby facilitating binding of the first leucine zipper domain to the second leucine zipper domain.

In embodiments, the first protein binding domain and the second protein binding domain may be any two protein domains capable of binding to each other with strong binding affinity. In embodiments, the equilibrium dissociation constant (K_{D}) of a first protein binding domain binding to a second protein binding domain may be less than 100 uM. The equilibrium dissociation constant (K_{D}), is defined herein is the ratio of the dissociation rate (K-off) and the association rate (K-on) of a first protein to a second protein (e.g. a first protein binding domain to a second protein binding domain, a peptide to the MHC II complex, etc). It is described by the following formula: K_{D}=K-off/K-on. In embodiments, the K_{D} of the first protein binding domain binding to a second protein binding domain may be less than 100 uM, 50 uM, 25 uM, 10 uM, 1 uM, 100 nM, 10 nM or 1 nM. In embodiments, the first protein binding domain and the second protein binding domain may be any two domains capable of forming covalent bonds (e.g. disulfide bonds). For example, the first protein binding domain and second protein binding domain may bind to form an Fc domain.

In embodiments, the first protein binding domain is attached to the C-terminus of the alpha chain and the second protein binding domain is attached to the C-terminus of the beta chain. In embodiments, the alpha chain is an HLA-DR alpha chain and the beta chain is an HLA-DR beta chain. In embodiments, the alpha chain is an HLA-DQ alpha chain and the beta chain is an HLA-DQ beta chain. In embodiments, the alpha chain is an HLA-DP alpha chain and the beta chain is an HLA-DP beta chain. In embodiments, the alpha chain is an HLA-DR4 alpha chain and the beta chain is an HLA-DR4 beta chain. In embodiments, the HLA-DR4 alpha chain has the amino acid sequence of SEQ ID NO:1. In embodiments, the HLA-DR4 beta chain has the amino acid sequence of SEQ ID NO:2. In embodiments, the alpha chain is an HLA-DQ6 alpha chain and the beta chain is an HLA-DQ6 beta chain. In embodiments, the HLA-DQ6 alpha chain has the amino acid sequence of SEQ ID NO:3. In embodiments, the HLA-DQ6 beta chain has the amino acid sequence of SEQ ID NO:4.

### Nucleic Acid Compositions

The MHC II complex provided herein including embodiments thereof may be expressed in a variety of methods known in the art. The MHC II complex may be encoded on RNA or DNA delivered to cells as a modified or unmodified RNA or plasmid DNA. The MHC II complex described herein, including embodiments and aspects thereof, may be provided as a nucleic acid sequence that encodes for the MHC II complex.

Thus, in an aspect is provided a nucleic acid encoding a major histocompatibility class II (MHC II) complex, wherein the MHC II complex includes an alpha chain and a beta chain, wherein the alpha chain is attached to a first protein binding domain and the beta chain is attached to a second protein binding domain, wherein the first protein binding domain and the second protein binding domain are capable of non-covalently binding to form a MHC complex. In embodiments, the nucleic acid further encodes a sequence encoding for a cell surface protein. In embodiments, the cell surface protein is Aga2p, a-agglutinin α-agglutinin, flocculin, Cwp1p, Cwp2p or Tip1p.

### Methods of Making

Provided herein, inter alia, are methods of making a major histocompatibility class II (MHC II) complex. In embodiments, the MHC II complex is expressed and covalently attached to the surface of a cell (e.g. cell surface display of the MHC II complex). Thus, the methods provided herein including embodiments thereof bypass the need for purification of the MHC II complex. The methods provided are contemplated to produce functional MHC II complex, wherein the MHC II complex recognizes MHC-II binding peptides.

Thus, in an aspect is provided a method of making a major histocompatibility class II (MHC II) complex, the method including transforming a cell with the nucleic acid provided herein including embodiments thereof, and culturing the cell under conditions wherein the MHC II complex is expressed. In embodiments, the cell is a yeast cell.

### Methods of Use

It is contemplated that the methods described herein may be used for identifying MHC II binding peptides. The methods provided herein include contacting a MHC II complex with a peptide, and detecting binding of the peptide to the MHC II complex. In embodiments, the MHC II complex is attached to the surface of a cell. The methods provided herein allow for screening of pathogenic peptides (e.g. from SARS-CoV-2) with large panels of MHC-II alleles (e.g. alleles from each of each of HLA-DR, HLA-DQ, and HLA-DP). For example, an array of cell clones, each expressing a unique MHC II complex (e.g. a MHC II allele) may be contacted with a peptide. Subsequently, peptide binding is detected, thereby allowing identification of the MHC II complex binding peptide.

The methods provided herein including embodiments thereof allow for analysis of MHCII allele variants by site-directed mutagenesis. In embodiments, the methods allow for directed evolution focusing on specific regions of MHCII protein. In embodiments, the methods provided herein allow for identification of MHC II complex binding peptides and its related allele variants. In embodiments, the methods provided herein allow for modification of the initial MHC II complex (e.g. site directed mutagenesis, directed evolution, etc.) to develop a stabilized MHC II complex. The stabilized MHC II complex may be used for crystallization studies, or generating stable peptide/MHCII tetramers for T cell staining.

Thus, in an aspect is provided a method of identifying a peptide that binds a major histocompatibility class II (MHC II) complex, the method including: i) contacting a cell provided herein including embodiments thereof with a peptide, and ii) detecting binding of the peptide to the MHC II complex, thereby identifying the MHC II complex binding peptide. In embodiments, the peptide includes a mixture of peptides. In embodiments, the peptide competes with a reference peptide for binding of the MHC II complex.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Background to Experiments Described Herein

CD4+ T cells orchestrate adaptive immune responses via ligation of their receptors for antigen by specific peptide/MHC-II complexes. To study these responses, it is essential to identify protein-derived, MHC-II peptide ligands that constitute immunodominant epitopes for T cell recognition. However, constructing single MHC II allele-expressing cells and isolating these proteins for use in peptide elution or binding studies is time-consuming. Here, we express human MHC alleles (HLA-DR4 and -DQ6) as native, non-covalent α/β dimers on yeast cells for direct, flow cytometry-based screening of peptide ligands from selected antigens. We demonstrate a rapid, accurate identification of DQ6 ligands from pre-pro-hypocretin, a narcolepsy-related immunogenic target. We also identified eleven DR4-binding, SARS-CoV-2 spike peptides homologous to SARS-CoV-1 epitopes and 2 spike peptides overlapping with reported SARS-CoV-2 epitopes recognized by CD4+ T cells from un-exposed individuals carrying DR4 subtypes. Our method is optimized for immediate application in the context of novel pathogens.

The peptide/MHC-II characterization pipeline (Rapid Identification of Peptide ligands from Protein Antigen (RIPPA)) allows elimination of the labor-intensive expression/purification steps by taking advantage of the yeast-display platform. As single-cell eukaryotes, yeast cells have the fast and easy cloning features of E. coli and are equipped with posttranslational modification machinery that is similar to mammalian or insect cells. We are the first, who used yeast to express native-like DR or DQ α/β heterodimers on the yeast cell surface and demonstrated their capability to directly screen peptide binders in a time-efficient manner. We linked one chain (α or β) of a given MHC-II allele to a yeast surface protein and allowed the other chain (α or β) to be secreted as a soluble component by the same yeast cell. We modified both C-termini of α/β; chains with leucine zipper motifs, which facilitate the pairing of the solubly secreted chain with the other surface-anchored chain. Our unique design for the surface expression of non-covalent MHC-II α/β heterodimers does not need covalently linked place-hold peptides, inter-chain linkers, or mutations of MHC-II amino acid residues. It results in correctly folded and fully functional MHCII proteins displayed on the yeast cell surface. The RIPPA assay we developed relies on the competitive binding between the tested peptide and a reference peptide to yeast-displayed MHCII protein.

With our invention of RIPPA, a much larger panel of MHC-II alleles can be assayed within a short time frame and in a cost-efficient manner. By creating an array of yeast cell clones, each expressing a particular MHC-II allele in their native-like format, RIPPA is particularly useful for quickly screening pathogenic peptides (i.e., from SARS-CoV-2) for their binding to tens to hundreds of MHC-II alleles within a month. Knowing relevant peptide/MHC-II characteristics will guide T cell research as well as downstream development of vaccines in emergency use against rapidly spreading diseases, like COVID-19. In addition to the empirical benefits, the fast generation of a huge amount of peptide/MHC-II binding data by RIPPA will advance the computational approaches aiming at T cell epitope discovery. Of note, many TCR start-ups are computationally rich and the training of their epitope prediction algorithms is highly dependent on peptide/MHC-II data.

### Example 2: Yeast Display of MHC-II Enables Rapid Identification of Peptide-Ligands from Protein Antigen (RIPPA)

### Introduction

CD4+ T cell responses are crucial drivers of defensive immunity against infection; however, they can also cause autoimmune responses when tolerance breaks down. These T cells respond to antigen via the interaction between the T cell receptor (TCR) and antigen-derived peptides bound to heterodimeric (a/(3) major histocompatibility complex class II (MHC-II) molecules on the surface of professional antigen-presenting cells (APCs)^{1, 2}. Identification of T cell responses specific for a target antigen requires intensive screening of overlapping peptides covering the candidate protein in a T cell assay. Using characterized peptide/MHC as probes for TCR binding or TCR-mediated T cell activation allows further assessment of reactive T cell clones. For example, we and others have applied both in vitro T cell assays and ex vivo peptide/MHC-II probing to prove the existence of CD4+ T cell clones targeting a neurotransmitter (hypocretin) in narcolepsy patients³⁻⁵. Recently, similar analyses have identified CD4+ T cell responses to SARS-CoV-2 viral proteins⁶⁻⁹, however, a lack of well-characterized MHC-II ligands derived from these viral antigens limits the interrogation of reactive T cell clones.

The T cell repertoire is tremendously diverse and TCRs expressed by distinct clones bear different specificities for particular peptides bound to particular MHC-II alleles^{10, 11}. Thanks to the development of MHC (class I and II) tetramer staining technology¹², one can stain and isolate peptide/MHC tetramer positive T cell clones for functional and structural investigations¹³⁻¹⁵. The current limitation is that tetramer synthesis requires information on the binding of MHC to peptides derived from the candidate protein antigen. Both computational and experimental efforts have been made to generate such binding information, with the former largely relying on empirical data¹⁶⁻¹⁹. There are two major experimental approaches to determine MHC-II ligands. One uses mass spectrometry to quantify peptides eluted from MHC-II molecules that are immunoprecipitated from lysed cells¹⁹⁻²². The other detects the binding of synthesized peptides to soluble, recombinant MHC-II proteins^{5, 13, 23-26} Currently, to identify peptides bound by a particular MHC-II allele, it is optimal for either method to generate cell lines that express only this allele, as primary cells in human are typically heterozygous and co-dominantly express both alleles from each of HLA-DR, -DQ, and -DP, the three isotypes of human leucocyte antigens (HLA). In addition, both methods require purification of the expressed MHC-II protein. These steps typically take up to 4 months and significantly limit the speed of empirical studies.

Here, we develop a new methodology that allows elimination of the labor-intensive expression/purification steps by taking advantage of yeast display²⁷. As single-cell eukaryotes, yeast cells have the fast cloning capability of E. coli and are equipped with post-translational modification machinery that is similar to mammalian cells^{27, 28}. Therefore, linking an exogenous protein (e.g., MHC-II) to a native yeast protein on the surface offers a fast way to investigate the function of the exogenous protein. In order to express MHC-II alleles, including DR and DQ, as non-covalent heterodimers without an inter-chain linker on the yeast cell surface, we replace the transmembrane domains with the leucine zipper dimerization motifs^{29, 30} to facilitate pairing of a/0 chains that are separately secreted by yeast using a bi-directional expression constuct^{31, 32}. We prove that both DR and DQ constructs are correctly folded without the necessity of covalently linked peptides and fully functional in binding exogenous peptides. We then design a competition assay that enables rapid identification of MHC-II peptide-ligands from protein antigen (RIPPA), using hypocretin and the SARS-CoV-2 spike (S) model antigen. The quick set-up time (<1 month) for the RIPPA in vitro peptide-binding allows efficient testing of MHC-II ligands to guide tetramer synthesis and expedite downstream investigation of cell-mediated immune responses that are relevant to disease. These characteristics are particularly useful in the setting of novel, rapidly spreading diseases, like COVID-19.

Results

Leucine Zippers Enhance MHC-II Expression on Yeast, Independent of Peptide Ligands

Professional APCs express MHC-II as α/β heterodimeric membrane proteins associated with the chaperone, invariant chain (Ii). The peptide binding groove of nascent MHC-II is occupied by a region of Ii that is proteolytically trimmed to yield the CLIP peptide³³. An antigenic peptide capable of binding to a given MHC-II allele can replace CLIP through a peptide exchange process. This process most often takes place in the MHC-II containing compartment (MIIC), where it is typically catalyzed by HLA-DM³⁴, although cell surface exchange may occur in some situations³⁵. It is likely that mass spectrometry underestimates certain MHC-II binders in the eluted ligandome when these binders are out-competed by high abundant or high affinity peptides due to physiologic (e.g., intracellular cleavage, DM effects) or experimental (including differences between model cell lines and primary cells) conditions. Therefore, to explore all possible MHC-II ligands from candidate antigen, it is ideal to evaluate MHC-II binding of overlapping peptides spanning the entire antigen. When recombinant MHC-II ectodomains are used in binding studies, a specific peptide ligand is typically linked to at the β N-terminus to stabilize α/β dimers. A linker removal step then is necessary to ensure an exchangeable placeholder peptide in a peptide-binding assay²³.

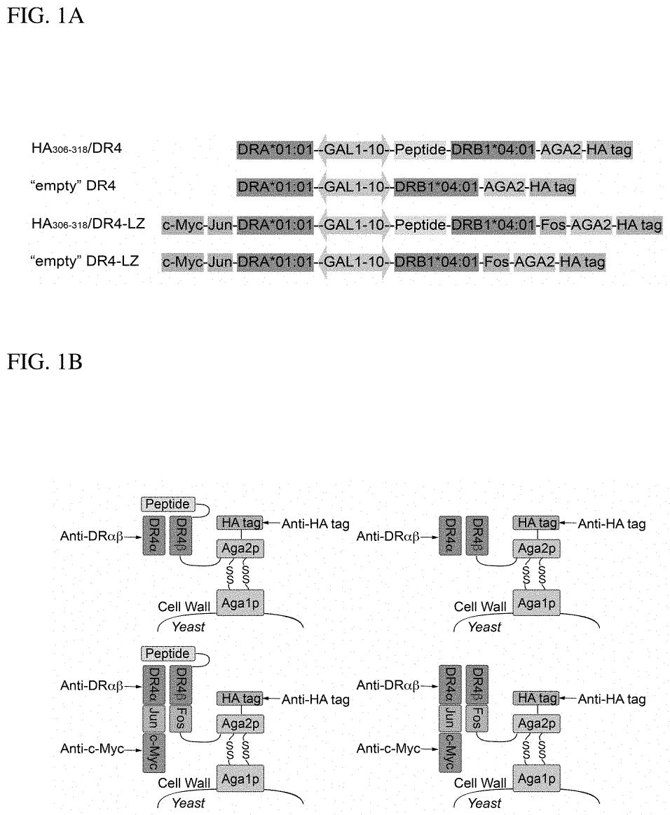

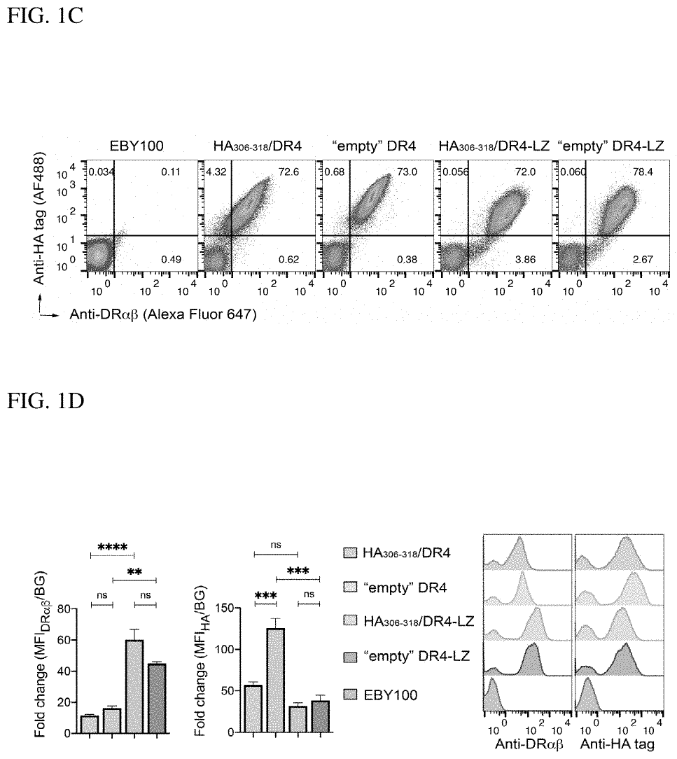

To avoid the linker removal step in peptide-binding using yeast display of MHC-II, we first utilized an “empty” construct expressed by yeast. We used DR4 (DRA*01:01/DRB1*04:01) as a representative DR allele, with an influenza hemagglutinin (HA)₃₀₆₋₃₁₈ peptide-linked construct that was previously examined in yeast³². Importantly, a bi-directional GAL1-10 promoter directs the simultaneous expression of α and β chains in a yeast shuttle vector (FIG. 1A). Unlike several previous attempts at yeast display using a single-chain format of recombinant DR proteins³⁶⁻³⁹, the non-covalent heterodimer format no longer requires mutations of MHC-II to facilitate protein folding thus enables binding and characterization of true MHC-II ligands. Considering the potential instability of the “empty” DR4 protein, we included leucine zippers (LZ) motifs^{29, 30} in two additional constructs to facilitate dimerization (FIG. 1A). LZ Fos/Jun motifs allowed α/β pairing of a single-chain DR protein in yeast despite it was not functional³⁷. For all four constructs, the DR4 β chain ectodomain, with or without the Fos motif, was engineered to the upstream of yeast AGA2 gene followed by an HA epitope tag, while the α chain ectodomain, with or without the Jun motif, was designed to be secreted from yeast (FIG. 1A). Successful folding and α/β pairing can yield functional DR4 expressed as a fusion to the yeast native surface protein agglutinin, composed of Aga1p and Aga2p subunits (FIG. 1B).

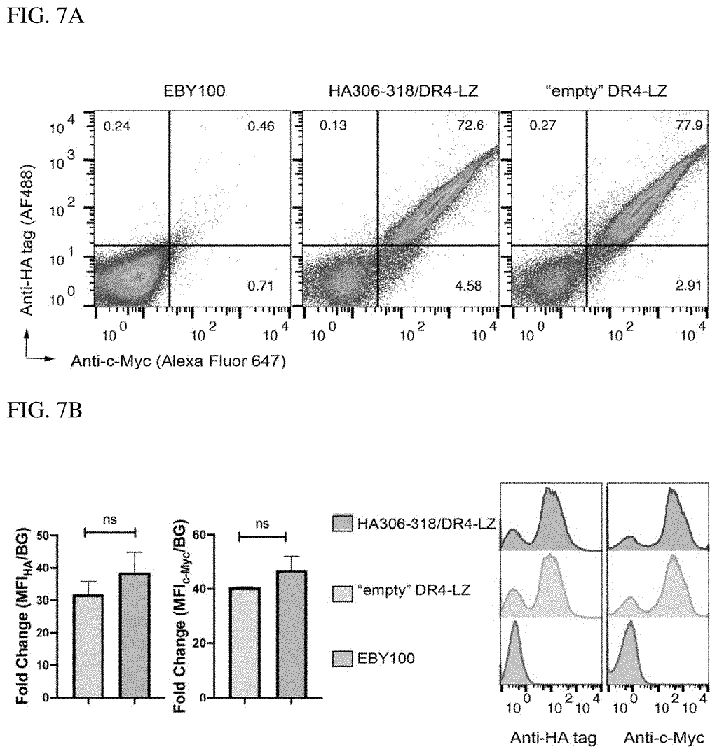

We created four yeast strains by individually transforming the above constructs into the parent Saccharomyces cerevisiae strain, EBY100²⁷. After induction of protein expression, we detected properly assembled DR4 on the surface of all four strains by co-staining with two antibodies, one specific for DRαβ (clone L243) and the other specific for the HA-tag (FIG. 1C). As expected, the LZ enhanced the surface level of folded DR4, 5× and 3× for the peptide-linked and “empty” constructs, respectively, as measured by flow cytometry (FIG. 1D). This improvement was attributed to α/β dimerization facilitated by LZ, and not yeast protein production, as levels of Aga2p expression, represented by staining of HA-tag, were not correlated with levels of folded dimers (FIG. 1D). Notably, L243 detected similar levels of “empty” DR4 compared to DR4 with linked peptides, implying that the “empty” binding grooves were occupied by peptides derived from the yeast culture³⁹, rather than truly empty (quotation marks indicate genetically empty). To further validate that both chains of DR4 in the LZ formats were expressed by yeast, we simultaneously stained the α and β chains. Co-staining of a c-Myc-tag at the C-terminus of α chain (FIG. 1A) and the HA-tag following DRβ-Aga2p fusion confirmed the expression of both the α- and β-encoding open reading frames (FIGS. 7A-7B). The presence of both chains on the surface is also consistent with the L243 antibody detection of correctly folded DRαβ (FIG. 1C).

Yeast Display of Peptide-Linked or “Empty” DQ Molecules as Non-Covalent Heterodimers

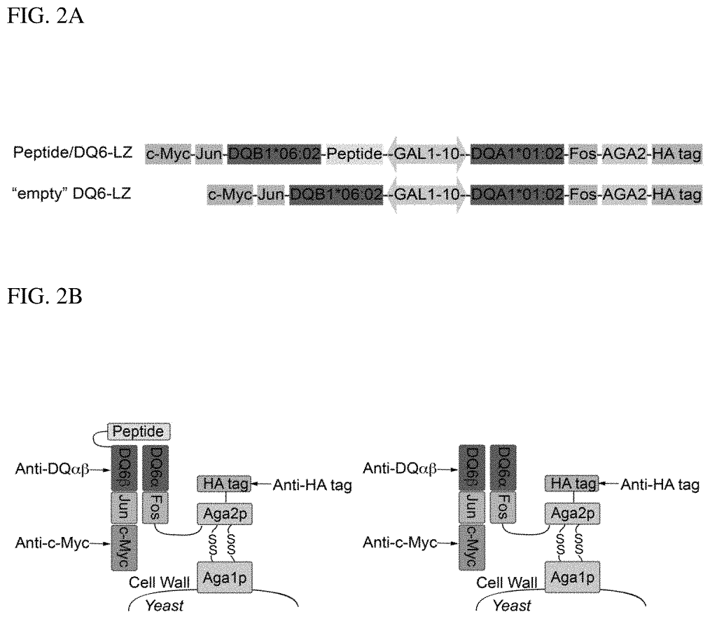

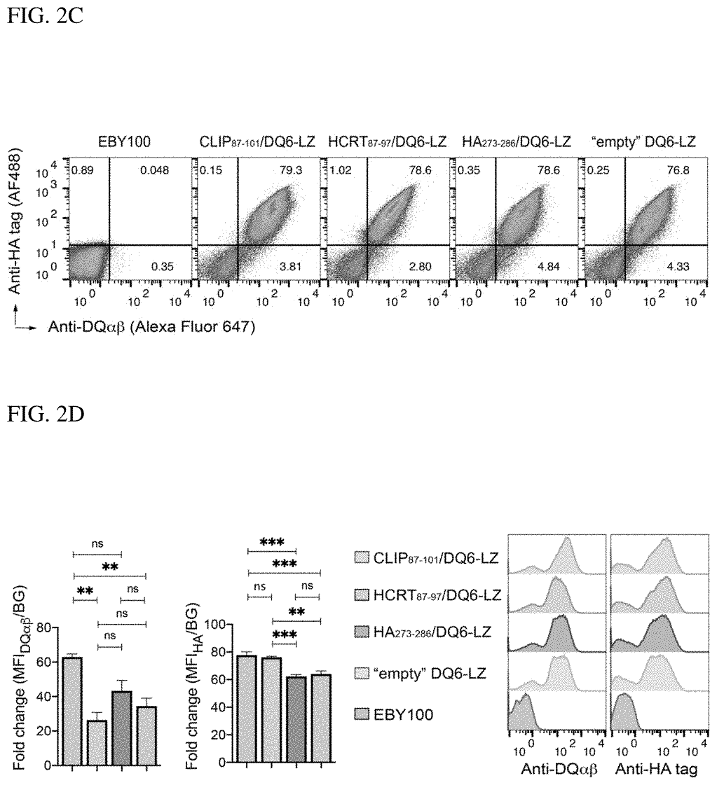

Next, we tested the expression of a representative DQ allele, DQ6 (DQA1*01:02/DQB1*06:02), on the yeast surface, using the same strategy as for DR4, except that the β chain was the secreted component (FIG. 2A). Successful chain pairing enables surface display of a functional DQ6 ectodomain (FIG. 2B). It is known that individuals carrying this DQ6 allele are susceptible to narcolepsy³⁻⁵, and during the 2009 flu pandemic, a significant increase in narcolepsy incidence occurred among DQ6+ populations, in association with natural infection or a particular vaccine formulation^{40, 41}. Several reports have identified DQ6-binding antigenic peptides in both the self-protein hypocretin (HCRT) and the viral proteins including haemagglutinin (HA) from the 2009 H1N1 influenza virus^{4, 5}. In peptide-linked constructs (FIG. 2A), we included the Ii peptide, CLIP₈₇₋₁₀₁, and two peptides, HCRT₈₇₋₉₇ and H1N1-HA₂₇₃₋₂₈₆, whose binding to DQ6 has been characterize^{4, 5, 42}.

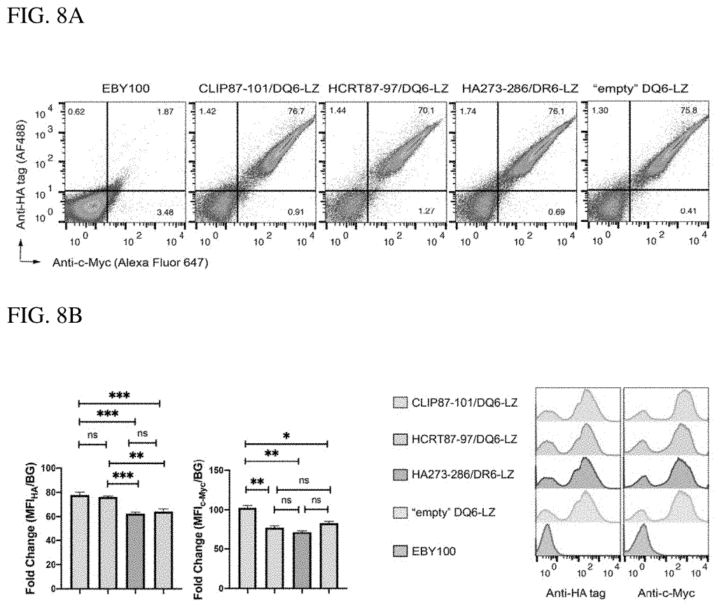

Similar to the DR4-expressing strains, after gene transformation and protein induction, all four yeast strains expressing the DQ6 constructs showed a double positive population, after surface co-staining with antibodies to the HA-tag and to a DQαβ conformational determinant, detected by mAb clone SPV-L3 (FIG. 2C). Notably, the levels of appropriately folded DQαβ varied significantly, with linked CLIP₈₇₋₁₀₁ giving the highest level (FIG. 2D). Co-staining of the c-Myc-tag and the HA-tag, as analyzed for DR4, further confirmed the expression of both DQ6 α and β ectodomains in yeast (FIGS. 8A-8B). Notably, the relative expression level of the secreted β chain represented by fold change of c-Myc-tag staining was significantly higher in the CLIP₈₇₋₁₀₁/DQ6-LZ construct, consistent with the fold change of DQαβ staining.

Collectively, both “empty” DR and DQ constructs yielded properly assembled MHC-IIαβ on the surface of yeast, facilitated by the LZ dimerization. Although the presence of linked peptides may influence protein expression, the successful display of “empty” MHC-II as a non-covalent heterodimer enabled further evaluation of their capability to accommodate exogenously added peptides.

“Empty” MHC-II on yeast binds specific peptides under various conditions

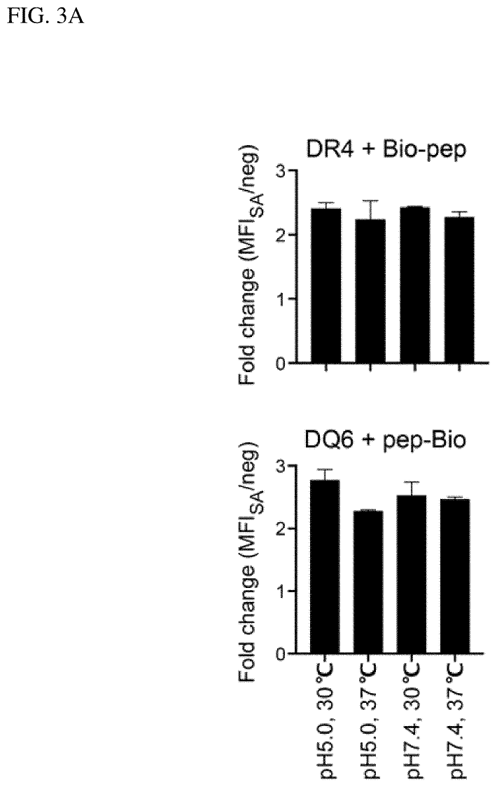

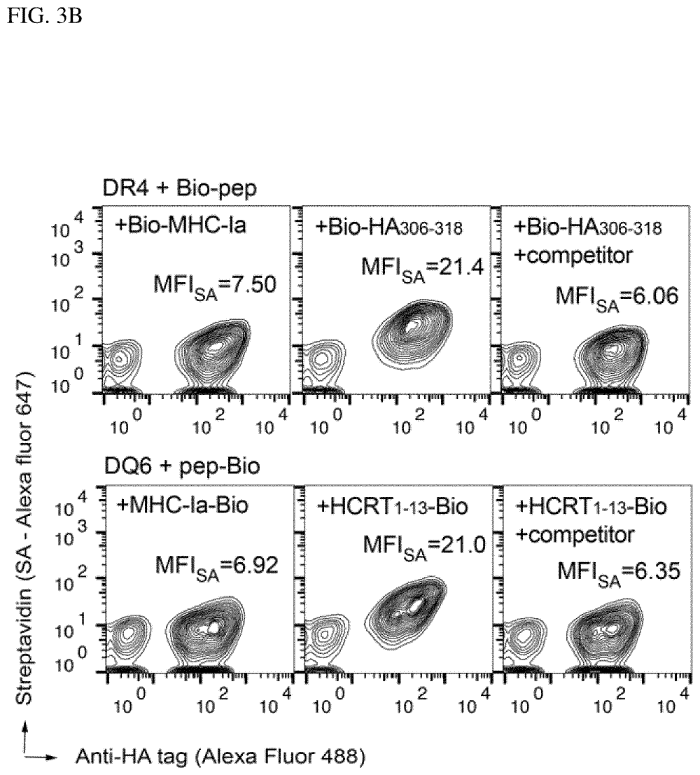

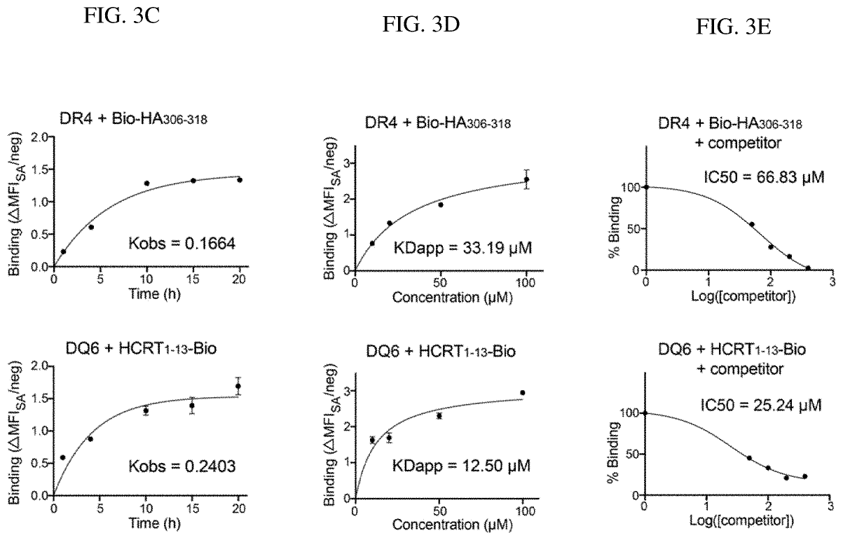

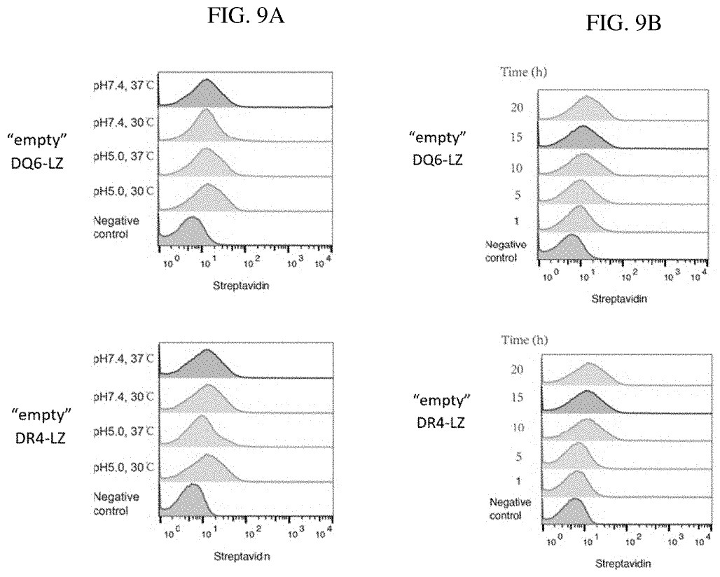

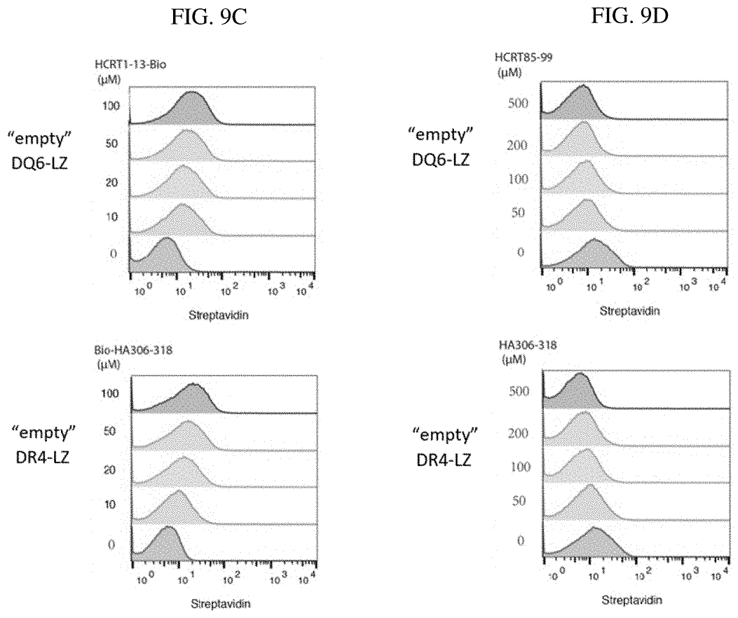

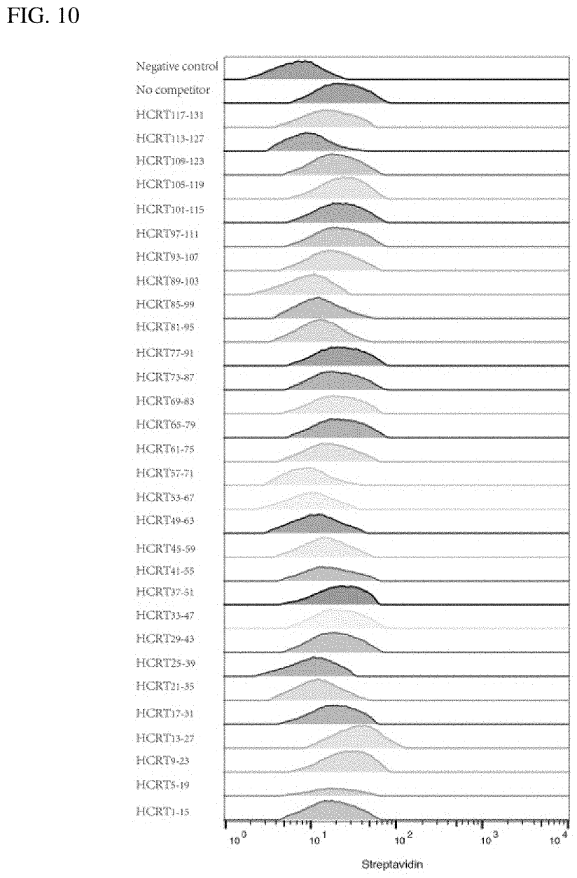

Physiologic peptide loading occurs in acidic MIIC (pH-5) at human body temperature (37° C.)³³. Yeast cells express proteins at 30° C. in a culture with pH ranging from 5 to 7. To demonstrate yeast display as a robust system to study MHC-II peptide binding, we examined various binding conditions. Yeast incubated with an indicator biotinylated peptide ligand consistently yielded 2-3× higher biotin signals compared to the irrelevant biotinylated peptide control at pH 5.0 vs pH 7.4 and at 30° C. vs 37° C. (FIGS. 3A-3B and FIG. 9A). The capacity to bind exogenous peptides validated the functional integrity of non-covalent MHC-II α/β heterodimers on yeast. We then determined the observed association rate constant (Kobs) for binding of the indicator peptide to “empty” MHC-II on yeast in a time course experiment (FIG. 3C and FIG. 9B). The kinetics are very similar to the ones we observed using soluble proteins in an peptide-loading assay²³ and suggest an apparent equilibrium at around 15-20 h. Incubation at pH 5.0 (physiologic binding pH), 30° C. (yeast habitual temperature) for 20 h was used for the KDapp measurement (FIG. 3D and FIG. 9C) and the downstream competitive binding studies. >20 μM of biotinylated indicator peptides were sufficient to stain the majority of cells (FIG. 3B and FIG. 9D).

We then asked whether non-biotinylated peptide ligands would competitively inhibit biotinylated indicator peptides if added to the yeast culture at an excess amount. A concentration-dependent inhibitory effect confirmed the competitive binding between non-biotinylated and biotinylated ligands to the MHC-II protein on yeast (FIG. 3B, FIG. 3E and FIG. 9D). The calculated IC50 within the range of 10-100 μM was consistent with the observation that >20 μM of peptide ligands were sufficient for loading to occur at the cell surface (FIG. 3D). The spontaneous peptide loading and competition suggests that “empty” MHC-II on yeast are pre-loaded with low affinity peptides that can be easily replaced by exogenous peptides, unlike our previous peptide loading of soluble DQ6 that requires the addition of soluble DM to catalyze the replacement of pre-bound CLIP²³. Notably, the micromolar KDapp and IC50 determined at yeast surface (FIGS. 3D-3E) are different from the nanomolar affinities observed in soluble MHC-II peptide binding, especially for DR4/HA₃₀₆₋₃₁₈ binding²⁶. However, the difference has no influence on the determination of relative binding capacities between competitor peptides to a given MHC-II. The competition assay thus offers a simplified and robust platform for rapid identification of unknown MHC-II ligands from a candidate protein antigen (RIPPA).

Similar Ligands Identified Using Yeast-Displayed Versus Soluble DQ6

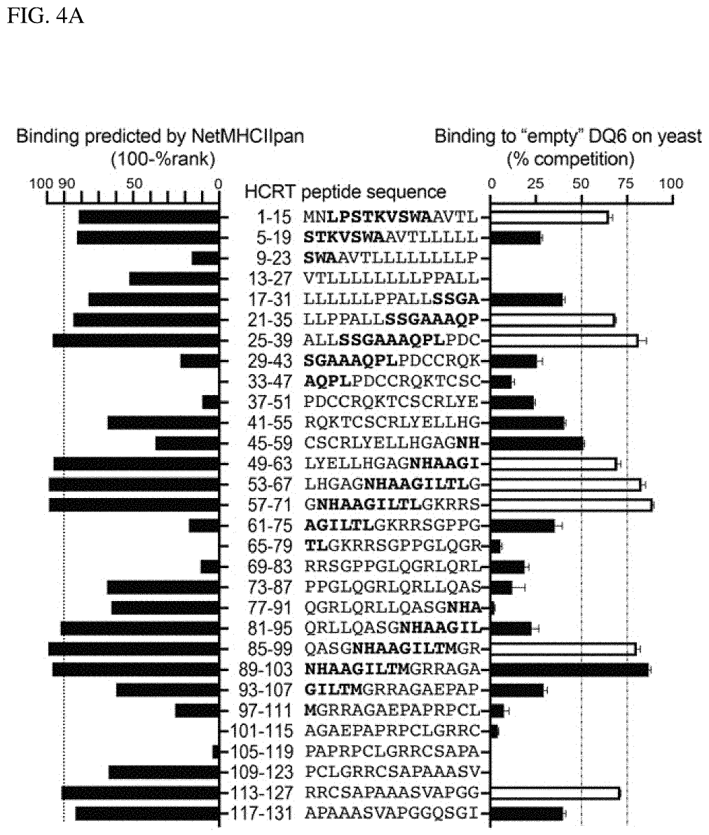

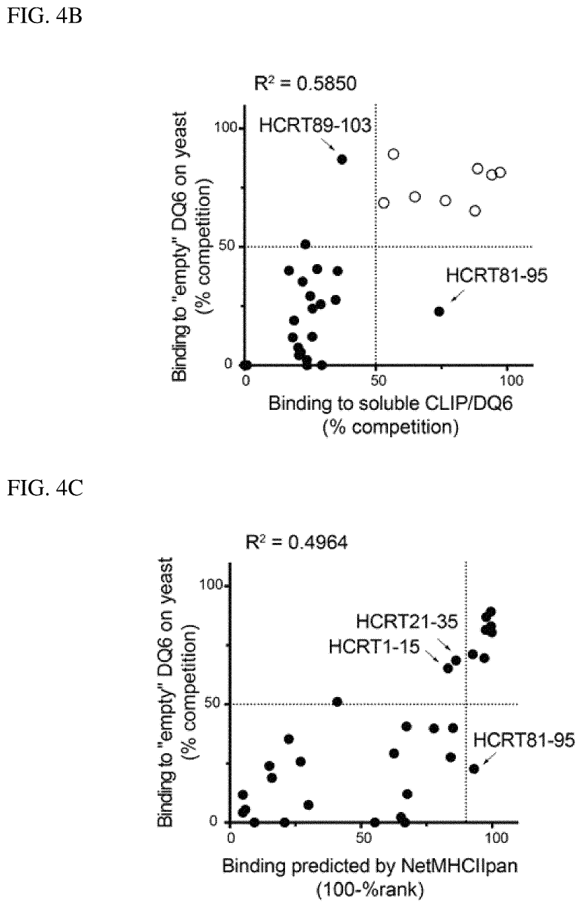

As a demonstration of the yeast-facilitated RIPPA methodology, we screened a set of 15-mer overlapping peptides (FIG. 4A) covering the hypocretin precursor (pre-pro-HCRT) for DQ6 ligands. As reported previously⁵, using the same set of overlapping peptides, we have identified five regions in the pre-pro-HCRT protein that generate peptides capable of binding to soluble DQ6 protein; we utilized the corresponding HCRT peptide/DQ6 tetramers to isolate in vivo expanded CD4+ T cells from narcoleptic patients and healthy controls. A comparison of the peptide binding data generated using yeast displayed DQ6 versus soluble DQ6 versus the computational prediction by the widely used NetMHCIIpan-4.0 server¹⁸ allowed us to examine the efficiency and accuracy of RIPPA (FIGS. 4A-4C). Unlike the “empty” DQ6 on yeast, the soluble DQ6 protein with linked CLIP₈₇₋₁₀₁ requires thrombin cleavage and DM catalysis for CLIP removal and peptide loading⁵. Despite different conditions, the two experimental results show a strong correlation (R²=0.5850, FIG. 4B), which is slightly higher than the correlation (R²=0.4964, FIG. 4C) between data acquired using the yeast display approach and predicted by NetMHCIIpan-4.0. Both experimental approaches identified HCRT₁₋₁₅, HCRT₂₁₋₃₅, HCRT₂₅₋₃₉, HCRT₅₃₋₆₇, HCRT₅₇₋₇₁ and HCRT₈₅₋₉₉ as DQ6 ligands (% competition>50%), whereas the RIPPA method further identified HCRT₈₉₋₁₀₃ (% competition>75%, FIGS. 4A-4B), yielding a 100% coverage of previously determined 9-amino-acid core registers^{4, 5, 42} However, HCRT₁₋₁₅ did not reach the top 10% rank, a default cut-off used by NetMHCIIpan-4.0 to suggest binders (FIGS. 4A-4C), thus missing the potential T cell epitope using register (LPSTKVSWA) that had been proven to bind DQ6 by X-ray crystallography⁴³. It is possible that HCRT₄₉₋₆₃ or HCRT₈₁₋₉₅, which cover <8 residues of the known 9aa core registers, bound DQ6 using different registers. Alternatively, the observed binding reflects the existence of false positives in all these methods, and on this occasion, the false positive rate is low in RIPPA (FIGS. 4A-4C). Overall, the comparison validates RIPPA as an efficient and accurate method in identification of DQ6 ligands derived from pre-pro-HCRT.

Identification of DR4 Peptide-Ligands from SARS-CoV-2 S Protein

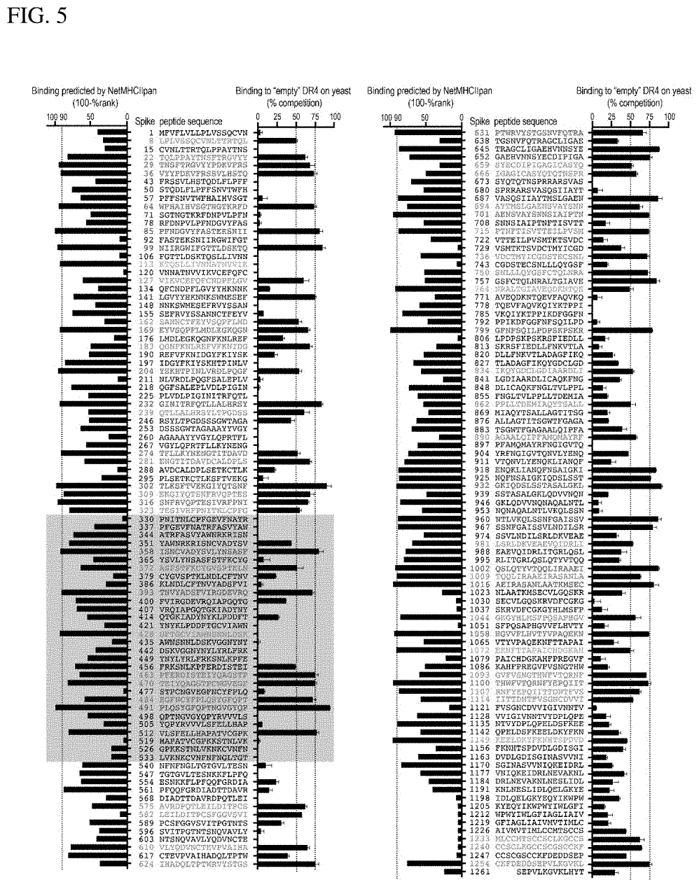

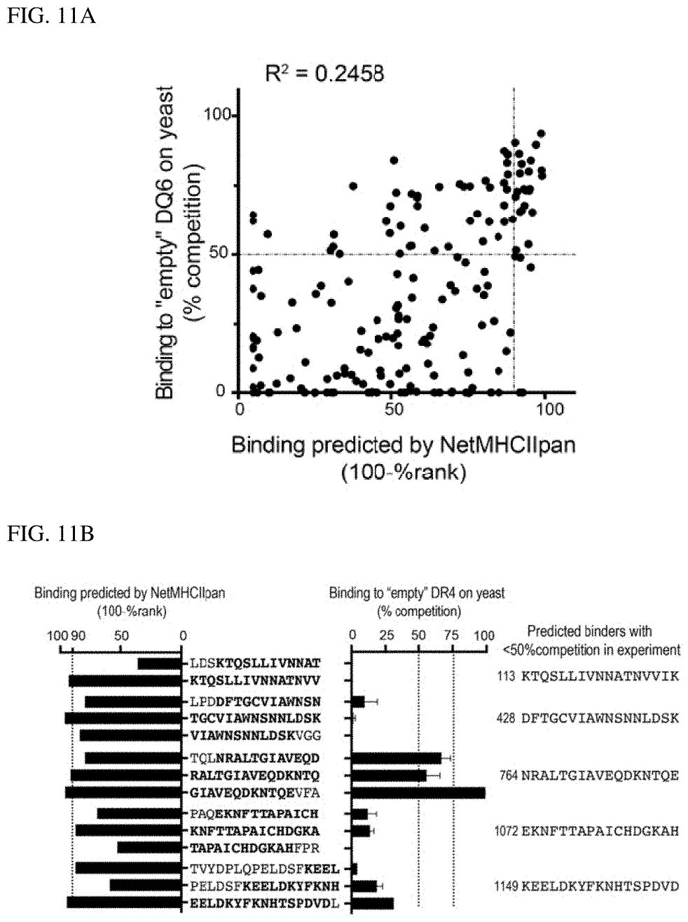

We next applied RIPPA to identify MHC-II ligands from the SARS-CoV-2 S protein. As a demonstration, we screened a set of 17-mer overlapping peptides (FIG. 5) for DR4 ligands. DRB1*04:01 has a frequency-10% in the population of European descent and 1-2% in other populations (https://bioinformatics.bethematchclinical.org). A 96-well PCR plate enabled simultaneous culture of 96 wells of sufficient yeast expressing “empty” DR4 and allowed screening for up to 94 non-biotinylated peptides as competitors (one per well) against the indicator peptide binding to the “empty” DR4 on yeast. This approach identified 20 strong and 47 weak DR4 binders (FIG. 5). 10/20 (50%) of these strong binders and 12/47 (25.5%) of these weak binders qualify as binders using the default 10% rank cut-off by NetMHCIIpan-4.0. The insufficient coverage of RIPPA-identified DR4 ligands by prediction is also reflected by the lower correlation (R²=0.2458, FIG. 11A) as compared with the data correlation observed for pre-pro-HCRT analysis (R²=0.4964, FIG. 4C). This likely reflects the aforementioned limitation that mass spectrometry underestimates certain binders after elution (EL), resulting a lower rank in predictions, like NetMHCIIpan-4.0 trained on both EL and binding affinity (BA) data. However, 36/45 (80%) of the RIPPA-identified DR4 ligands have relatively high rank (top 11-50%) with some near the top 10% rank (FIG. 5), indicating that NetMHCIIpan-4.0 still represents one of the useful computational platforms. We noticed that 5 predicted DR4 binding 17-mer peptides were poor competitors (50% competition) in the RIPPA analysis. To eliminate experimental error caused by a selection of certain peptides, we synthesized alternative peptides spanning these five 17-mer peptide regions and tested their competitive binding to “empty” DR4 on yeast. Only 3 of these alternative peptides spanning 5764-780 showed 55-99% competition (FIG. 11B), suggesting S764-780 as a DR4 ligand, although its competition is slightly <50% in RIPPA. Alternative peptides spanning the other four regions showed 0-33% competition, confirming that the corresponding 17-mers are false positives by prediction (FIG. 11B).

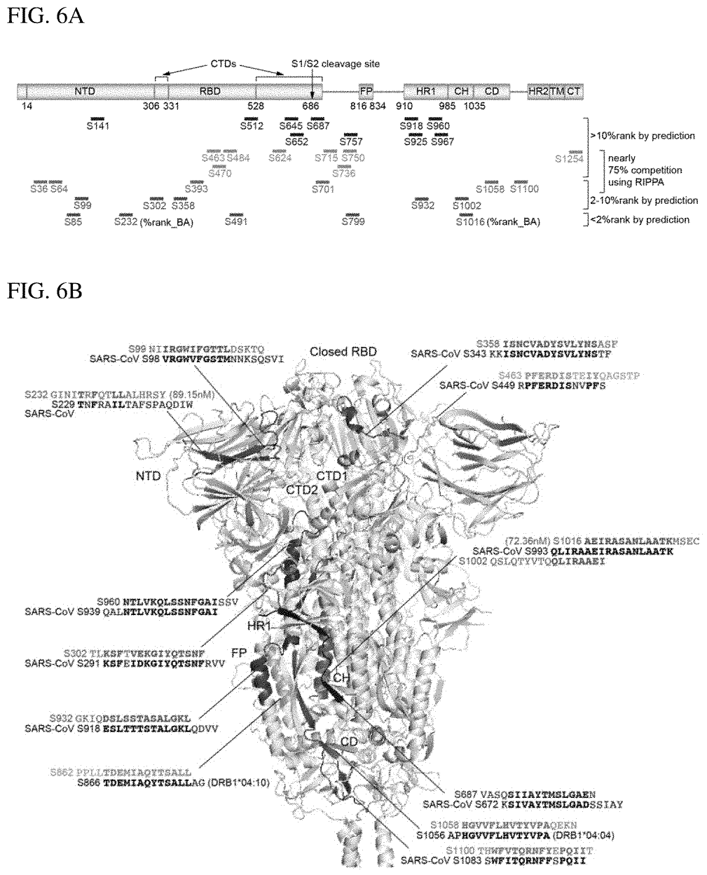

We next located RIPPA-identified DR4 ligands in different domains of the S protein. RIPPA identified 38 DR4 ligands from the 51 subunit and 30 from the S2 subunit (FIG. 5). 51 contains an N-terminal domain (NTD), two C-terminal domains (CTD1 and 2) and the receptor-binding domain (RBD), which is essential for viral attachment and transmission⁴⁴. The S2 subunit, including a fusion peptide (FP), two heptad repeats (HR1 and HR2), a central helix (CH) and a connector domain (CD), functions to bring viral and cellular membranes into close proximity for fusion and infection⁴⁵. As physiologic DM catalysis and peptide editing inside MIIC selects high affinity peptides for MHC-II presentation³⁴, we have particular interest in strong binders that have a high likelihood of surviving intracellular regulation in APCs and being recognized by CD4+ T cells. All strong binders are from the S ectodomain and there is no particular location preference, suggesting a wide range of candidate immunogenic targets for vaccine design (FIG. 6A).

Fifteen DR4-restricted SARS-CoV-1 S peptides have been previously suggested to be putative T cell epitopes¹³; 10/15 (66.7%) of these SARS-CoV-1 S epitopes share at least 4aa homology with eleven SARS-CoV-2 S peptides that are either strong DR4 binders or show nearly 75% competition in RIPPA (FIG. 6B). Although 3/11 (27.3%) of these peptides rank lower than top 10% (>10% rank) by NetMHCIIpan-4.0 prediction, S232-248 and S1016-1032 are predicted to have high DR4 binding affinities, 89.15 nM and 72.36 nM, respectively, and % rank_BA by NetMHCIIpan-4.0 above top 2% (FIG. 6B). Another two DR4 ligands, 5862-888 and S1058-1074, with nearly 75% competition in RIPPA (one >10% rank by NetMHCIIpan-4.0 prediction, FIG. 5 and FIG. 6A), each share 13aa residues with two SARS-CoV-2 S epitopes that can stimulate CD4+ T cells isolated from un-exposed individuals, carrying DR4 allelic variants⁶. We note the homology and overlap of RIPPA-defined DR4 ligands with SARS-CoV-1 or SARS-CoV-2 S-derived T cell epitopes. These epitopes provide the molecular basis for the speculation, stemming from recent studies⁶⁻⁹, that cross-reactive CD4+ memory T cells likely arose prior to the COVID-19 pandemic. Therefore, RIPPA identifies an initial set of DR4-restricted epitope candidates for peptide/DR4 tetramers to probe cross-reactive CD4+ T cell clones from DR4+ individuals.

Discussion

The global COVID-19 health emergency has led to intensive efforts to understand CD4+ T cell-mediated immunity against SARS-CoV-2″ for strategic guidance of vaccine design and immunotherapy approaches. Computational algorithms that have been trained on existing peptide elution and binding data serve as the quickest way to predict MHC-II ligands derived from candidate SARS-CoV-2 antigens for use in T cell assays^{46, 47} Given the false positive rate¹⁶⁻¹⁹ and incomplete coverage of ligands from selected antigens in prediction, as observed here, it is still essential to experimentally determine MHC-II binding and verify HLA restriction of T cell epitopes prior to downstream experimental and clinical investigations. In this study, we develop a RIPPA method to quickly interrogate binding of the spectrum of peptides from an antigen to a given MHC-II allelic protein displayed by yeast cells. Unlike other experimental set-ups, this method does not require time-consuming steps, including the construction and preparation of cell lines expressing a single target MHC-II allele and the labor-intensive isolation and preparation of MHC-II proteins.

The engineering of MHC α and ρ polypeptides as a single-chain fusion to Aga2p in yeast was previously developed, with the aim of establishing a high-throughput surface display platform to study MHC-peptide-TCR interaction. However, in most situations, point mutations in MHC and covalently linked stabilizer peptides are necessary for the appropriate protein folding^{14, 15, 36-39, 48}. These genetic modifications largely impede the application of this platform to the study of true MHC ligands, although mutated or modified MHC (mostly class I) proteins have been applied to explore the mimotope repertoire of interesting TCRs^{14, 15, 38}. In order to display MHC-II in its native heterodimeric form, which is capable of peptide occupancy, we adopted a bi-directional expression construct^{31, 32} that enables α and ρ chains to be expressed separately in yeast. We also utilized a previously verified LZ dimerization motif^{29, 30} to facilitate chain pairing. Our construct yields “genetically empty” yet correctly folded DR or DQ α/β heterodimers anchored at the yeast cell surface to mimic their native counterparts for binding antigenic peptides. Both “empty” DR4 and DQ6 proteins are fully functional and accommodate biotinylated indicator peptides under various conditions, allowing both the optimum time and pH for MHC-II peptide binding and the habitual temperature for yeast survival. Also convenient is that molecules like DQ6 that typically rely on DM catalysis to remove a pre-loaded stabilizer peptide (e.g., CLIP), can spontaneously bind the biotinylated indicator peptide on yeast and be detected using flow cytometry. As described before^{31, 39, 49} and demonstrated here, the flow cytometric measurements can be mathematically converted to determine binding characteristics, and, importantly, the relative binding of different peptides. This feature enables the study of competitive peptide binding at the yeast cell surface and the development of a scalable RIPPA approach for identification of MHC-II ligands. Here, we validated the efficiency and accuracy of RIPPA and then identified DR4 ligands from the SARS-CoV-2 S protein as a model antigen.

RIPPA identified DR4 binding peptides from several major domains of the SARS-CoV-2 S protein, some of which have been suggested to be CD4⁺ T cell epitopes in a recent study, though their HLA restriction was not empirically defined⁶. Notably, more than half of these ligands rank below the default cut-off, as predicted by a widely used computational tool. There are also at least 4 false positives present in the predicted epitopes, reemphasizing the importance of empirical examination of MHC-II ligands. Knowing the capacity of MHC-II to bind peptides derived from multiple domains of a microbial antigen, as demonstrated here for DR4 ligands, provides useful information for vaccine design, particularly for subunit- or peptide-based agents. Viruses, including coronaviruses, mutate naturally for increased fitness. Notably, SARS-CoV-2 has given rise to potentially more pathogenic strains since first discovered, and currently the most prevalent mutation is an amino acid change at position 614 from aspartate to glycine (D614G)⁵⁰. Therefore, combinatorial inclusion of alternative epitopes or selection of candidates with conserved amino acids across mutants or across different strains of the same species of viruses are optimal for vaccination.

The yeast display platform is also amenable to coupling with techniques such as directed evolution³⁸, single-cell sequencing¹⁰, and TCR-signaling assays¹¹ for characterization of TCRs and T cell epitopes or mimotopes, given the ability of yeast to display both “empty” and peptide-linked constructs of non-covalent MHC-II α/β heterodimers. In addition, it remains possible that these native-like MHC-II on yeast can support antigen presentation to CD4+ T cells, as previously tested³⁶, for potential development of “artificial” APCs for scientific and therapeutic purposes.

### Example 3: Materials and Methods

Methods

Materials: The plasmids Z47 and ptDR1 were gifts received from Dr. Eric Boder (University of Tennessee, Knoxville)^{31, 32} HotStarTaq DNA Polymerase was purchased from Qiagen (Valencia, Calif.). Vent® DNA Polymerase, restriction enzymes, DNA ligase, and DH5alpha Competent E. coli were from New England Biolabs (NEB, Beverly, Mass.). Oligonucleotides used as polymerase chain reaction (PCR) primers were synthesized by Integrated DNA Technologies, Inc. (IDT, Coralville, Iowa). The DNA sequencing service was provided by MCLAB (South San Francisco, Calif.). Four biotinylated peptides: Bio-HA₃₀₆₋₃₁₈ (biotin-Ahx-PKYVKQNTLKLAT), Bio-MHC-Ia (biotin-Ahx-APWIEQEGPEYWDQE)⁵¹, HCRT1-13-Bio (MNLPSTKVSWAAVK-Ahx-biotin) and MHC-Ia-Bio (APWIEQEGPEYWDQEK-Ahx-biotin), and a non-biotinylated HA₃₀₆₋₃₁₈ were synthesized by GenScript (Piscataway, N.J.). The thirty 15-mer overlapping peptides (offset by 4aa, FIG. 4A) derived from prepro-HCRT were synthesized by GenScript. The 181, 17-mer overlapping peptides (the last one has 13aa, offset by 7aa, FIG. 5) derived from the SARS-CoV-2 S protein were ordered from BEI Resources (https://www.beiresources.org). Other tested spike peptides (FIGS. 11A-11B) were synthesized by Apeptide Co. Ltd (Shanghai, China). Monoclonal antibodies (mAbs), mouse anti-DRαβ (clone L243), mouse anti-DQ4 (clone SPV-L3) were affinity purified from ascites, as described previously^{31, 32} Rabbit anti-HA-tag mAb was purchased from Sigma (St. Louis, Mo.). Mouse anti-c-Myc-Tag mAb was purchased from Cell Signaling. Alexa Fluor 647 conjugated streptavidin was purchased from Invitrogen. Highly cross-adsorbed secondary antibodies, including Alexa Fluor 488 goat anti-rabbit IgG(H+L) and Alexa Fluor 647 goat anti-mouse IgG(H+L), were purchased from Thermo Scientific. Other chemical reagents were purchased from Thermo Scientific (Waltham, Mass.), unless indicated.

Creation of Yeast Display Constructs

The nucleotides encoding the HA₃₀₆₋₃₁₈ peptide in Z47 was first removed to construct a plasmid that allows the expression of “empty” DR4 in yeast (plasmid synthesis by GenScript). To construct “empty” DR4-LZ and HA₃₀₆₋₃₁₈/DR4-LZ plasmids, the Fos and Jun leucine zipper dimerization motifs as used previously^{5, 23, 37} were fused to the C-terminus of the DR4α and DR4β chain, respectively. The c-Myc epitope tag was added to the C-terminus of the Jun motif (plasmid synthesis by GenScript). The backbone yeast shuttle vector used for surface expression of “empty” DQ6-LZ or peptide/DQ6-LZ was based on the plasmid ptDR1, constructed previously³¹. A PCR fragment carrying the extracellular domain of HLA-DQA1*0102 with C-terminal Fos was cloned into ptDR1 in place of the original expression cassette coding for DR1 β chain via XmaI and SpeI restriction sites. This created an in-frame fusion of DQA1 to the N-terminus of Aga2p (pDQ6α). A second PCR fragment carrying the extracellular domain of HLA-DQB1*0602 with C-terminal Jun was cloned into pDQ6α in place of the original expression cassette coding for DR1 α chain via EagI and SalI restriction sites, to create ptDQ6-LZ that directs the expression of “empty” DQ6-LZ in yeast. Plasmids directing expression of peptide/DQ6-LZ constructs, including CLIP₈₇₋₁₀₁/DQ6 (CLIP₈₇₋₁₀₁ aa: PVSKMRMATPLLMQA), HA₂₇₃₋₂₈₆/DQ6 (HA₂₇₃₋₂₈₆ aa: RALLARSHVERTTD), and HCRT₈₇₋₉₇/DQ6 (HCRT₈₇₋₉₇ aa: SGNHAAGILTM), were synthesized (CLIP₈₇₋₁₀₁/DQ6-LZ and HA₂₇₃₋₂₈₆/DQ6-LZ constructs by GenScript) using a similar strategy, with the peptide sequence located upstream of the DQ6 β gene.

Protein Expression in Yeast

The plasmids carrying the tryptophan nutrition marker gene (TRP+) were then transformed into the yeast parent strain, EBY100 (URA+, TRP−), by electroporation following the BioRad MicroPulser protocol. After 2 days at 30° C., single yeast colonies can grow on agar plates containing tryptophan dropout medium, e.g., SD-CAA (2% w/v glucose, 0.67% w/v yeast nitrogen base without amino acids, 0.062% w/v Ura/Trp dropout casamino acids, 38 mM Na₂HPO₄, 62 mM NaH₂PO₄, pH 6.0). 2 ml of SD-CAA minimal media were then inoculated with a single yeast colony and cultured with 225 rpm shaking overnight at 30° C. to an OD₆₀₀ of 2.5-5.0. To induce GAL1-10-driven protein expression in yeast, 10⁷ cells were harvested and switched to 2 mL SG-CAA medium (glucose replaced by galactose). After 18 hours of induction at 30° C., sufficient yeast cells per sample were collected by centrifugation at 2,500 g for 3 min and washed and prepared for analysis of protein expression or peptide binding.

Immunofluorescent Staining and Flow Cytometry

The expression of “empty” or peptide-linked MHC-II was assessed using immunofluorescent labeling by flow cytometry. Briefly, galactose-induced yeast cells were first co-stained with primary mAbs including mouse mAb L243 (for DR4) or SPV-L3 (for DQ6) and rabbit anti-HA-tag mAb (˜10 μg/ml for each mAb) at room temperature (RT) for 30 min, then on ice for 30 min. Cells were then washed with 300 μl ice cold PBS+1% w/v bovine serum albumin (BSA) and double-labeled with highly cross-adsorbed secondary antibodies (at 1:100 dilution), Alexa Fluor 488 goat anti-rabbit IgG(H+L) and Alexa Fluor 647 goat anti-mouse IgG(H+L) on ice for 1 hour. To further validate that both chains of MHC-II were expressed by yeast, the mouse anti-c-Myc-Tag mAb (at 1:500 dilution) and rabbit anti-HA-tag mAb was used in the primary labeling step. After labeling, yeast cells were analyzed on a FACSCalibur flow cytometer (Becton, Dickinson and Company, Franklin Lakes, N.J.) to detect fluorescent signals corresponding to the expression of MHC-II proteins or an epitope tag. At least 100,000 cell events, gated by forward and side scatter, were collected per sample. Flow cytometric data was analyzed using FlowJo software (Version 10.6.0, BD).

Loading Exogenous Peptides to “Empty” MHC-II on Yeast

6×10⁵ galactose-induced yeast cells expressing “empty” MHC-II were collected by centrifugation at 2,500 g for 3 min and resuspended in 40 μl of the following solutions: citrate buffer (40 mM citric acid and sodium citrate, pH 5.0, 150 mM NaCl, 1% w/v BSA) and phosphate-buffered saline (PBS; pH 7.4, 137 mM NaCl, 2.7 mM KCl, 10.1 mM Na₂HPO₄, 1.8 mM KH₂PO₄, 1% w/v BSA). Biotinylated peptides, Bio-HA₃₀₆₋₃₁₈ and Bio-MHC-Ia for DR4 or HCRT₁₋₁₃-Bio and MHC-Ia-Bio for DQ6, were then added to the solution at 20 μM prior to incubation at the desired condition. To determine the kinetics of peptide binding, 6×10⁵ galactose-induced yeast cells were collected and resuspended in 40 μl 40 mM citrate buffer (pH 5.0) and incubated with biotinylated peptides at different time point, then collected by centrifugation at 2,500 g for 3 min for analysis by flow cytometry. To determine the apparent binding affinity of MHC-II peptide binding at the yeast cell surface, 0, 10, 20, 50, 100, μM of Bio-HA₃₀₆₋₃₁₈ and Bio-MHC-Ia for DR4 or HCRT₁₋₁₃-Bio and MHC-Ia-Bio for DQ6 were used to incubate with yeast cells in the 40 mM citrate buffer (pH 5.0) at 30° C. for 20 hours. Unrelated biotinylated peptides, MHC-Ia-Bio and Bio-MHC-Ia, were used as negative controls. The reaction tubes were sealed with parafilm before the incubation to prevent variation of culture volumes that may affect the final concentration of peptides in the time course or concentration titration studies. After incubation, the yeast cells were washed twice with 300 μl ice cold PBS+1% BSA before staining with streptavidin-AF647 diluted 1:200 in 50 μl PBS+1% BSA on ice for one hour. Cells were then washed with 300 μl ice cold PBS+1% BSA twice and finally resuspended in 300 μl ice cold PBS+1% BSA for analysis on a BD FACSCalibur flow cytometer (BD Biosciences). For simultaneous detection of both cell-surface MHC-II proteins and biotinylated peptides, cells with bound biotinylated peptides were first stained with rabbit anti-HA-tag mAb on ice for 30 min and then double-labeled with highly cross-adsorbed Alexa Fluor 488 goat anti-rabbit IgG(H+L) and streptavidin-AF647 in PBS+1% BSA on ice for one hour. Flow cytometric data was analyzed using FlowJo. The peptide binding was quantified as normalized median fluorescence intensity of the streptavidin staining signal, (MFI_{SA,indicator}−MFI_{SA,neg})/MFI_{SA,neg}, and plotted against the incubation time or the peptide concentration for determination of kinetic or thermodynamic parameters. To determine the observed association rate constant (Kobs) for binding of biotinylated peptides to “empty” MHC-II molecules at the yeast cell surface, data were fitted using nonlinear regression with the equation of one phase association in Graphpad Prism. To determine the apparent equilibrium dissociation constant (KDapp) for a biotinylated peptide binding to “empty” MHC-II molecules at the yeast cell surface, data were fitted using nonlinear regression with the equation of one site specific binding in Graphpad Prism.

Peptide Competition Assay Using Yeast Displaying MHC-II

6×10⁵ galactose-induced yeast were incubated at pH 5.0, 30° C. for 20 hours with 20 μM biotinylated indicator peptides in the presence of various concentrations of a competitor peptide to determine an appropriate competitor concentration for a competition assay. The non-biotinylated competitor peptides used for DR4 and DQ6 are HA₃₀₆₋₃₁₈ and HCRT₈₅₋₉₉, respectively. After incubation, the yeast cells were washed with PBS+1% BSA, stained with streptavidin-AF647 and analyzed by flow cytometry as described above. The % binding in the presence of competitors was quantified as [(MFI_{with competitor}−background)/(MFI_{without competitor}−background)]×100%, and plotted against the competitor concentration for determination of the half maximal inhibitory concentration (IC50). Data were fitted using nonlinear regression with the equation of one site fit log IC50 in Graphpad Prism.

Identification of DQ6 Binding Peptides in HCRT

Sufficient amounts of galactose-induced yeast cells displaying “empty” DQ6 were collected by centrifugation at 2,500 g for 3 min and resuspended in the 40 mM citrate buffer (pH 5.0) at a density of 1.5×10⁴ cells μl⁻¹. 20 μM HCRT₁₋₁₃-Bio peptide were then added to make a master mix of the reaction solution. 40 μl aliquots were aspirated from the solution, and each aliquot was supplemented with a 15-mer peptide (each at 200 μM) derived from the prepro-HCRT. In parallel, yeast without biotinylated peptides and yeast with MHC-Ia-Bio were also prepared and served as background and negative controls, respectively. The reaction was carried out under acidic conditions, at 30° C. for 20 hours. DQ6-associated HCRT₁₋₁₃-Bio was labeled with streptavidin-AF647 and analyzed by flow cytometry as described above. The binding of a competitor peptide to “empty” DQ6 on yeast was quantified as % competition=100%−[(MFI_{with competitor}−background)/(MFI_{without competitor}−background)]×100%.

High-throughput identification of DR4 binding peptides in SARS-CoV-2 spike protein

Here, the competitive binding assay is scaled to a 96-well format. Sufficient amounts of galactose-induced yeast cells displaying “empty” DR4 were mixed with 20 μM Bio-HA₃₀₆₋₃₁₈ in the 40 mM citrate buffer (pH 5.0) at a density of 1.5×10⁴ cells μl⁻¹. 40 μl of the master mix were dispensed into each well of a 96-well PCR plate (Eppendorf Twin.tec® PCR Plate 96) using multichannel pipettes except for the negative control and the well only with galactose-induced yeasts. Up to 94 synthesized non-biotinylated peptides derived from the SARS-CoV-2 S protein were then added into wells of a 96-well plate to a final concentration of 200 μM (one peptide per well). The reaction plate was sealed with plate sealer (Eppendorf Storage Foil) to prevent variation of culture volumes that may affect the concentration of peptides before incubation of the plate at 30° C. for 20 hours. After incubation, the yeast cells in the 96-well PCR plate were washed three times with 150 μl ice cold PBS+1% BSA using multichannel pipettes before staining with streptavidin-AF647 diluted 1:200 in 50 μl PBS+1% BSA on ice for one hour. Cells were then washed with 150 μl ice cold PBS+1% BSA three times and finally resuspended in 300 μl PBS+1% BSA for flow cytometric analysis. The binding of a competitor peptide to “empty” DR4 on yeast was quantified as % competition=100%−[(MFI_{with competitor}−background)/(MFI_{without competitor}−background)]×100%.

Comparison Between Experimental Data and NetMHCIIpan-Predicted Data

NetMHCIIpan-4.0 (http://www.cbs.dtu.dk/services/NetMHCIIpan) was used to predict binding of a HCRT or SARS-CoV-2 S peptide to MHC-II proteins. The new NetMHCIIpan-4.0 server is trained on both peptide elution (EL) and soluble MHC-II peptide binding (binding affinity, BA) datasets. NetMHCIIpan-4.0 server provides prediction scores for binding affinity, % rank_BA. However, we only used it for comparison when a peptide was experimentally determined to bind the MHC-II protein, as the binding affinity data are still limited for the training of NetMHCIIpan and accurate prediction. Therefore, unless specifically indicated, % rank scores that we cite in this study represent the likelihood of presentation by MHC-H, rather than the rank of relative binding affinity. Correlation between two sets of data was analyzed by plotting one set against the other on a xy-plot and R squared value was determined, using correlation analysis in GraphPad Prism. Higher correlation (represented by R square) indicates higher chance of the same peptide being an MHC-II ligand (or not), as determined by both datasets.

### REFERENCES

Rossjohn, J. et al. T cell antigen receptor recognition of antigen-presenting molecules. Annu Rev Immunol 33, 169-200 (2015).
La Gruta, N. L., Gras, S., Daley, S. R., Thomas, P. G. & Rossjohn, J. Understanding the drivers of MHC restriction of T cell receptors. Nat Rev Immunol 18, 467-478 (2018).
Latorre, D. et al. T cells in patients with narcolepsy target self-antigens of hypocretin neurons. Nature 562, 63-68 (2018).
Luo, G. et al. Autoimmunity to hypocretin and molecular mimicry to flu in type 1 narcolepsy. Proc Natl Acad Sci USA 115, E12323-E12332 (2018).
Jiang, W. et al. In vivo clonal expansion and phenotypes of hypocretin-specific CD4(+) T cells in narcolepsy patients and controls. Nature communications 10, 5247 (2019).
Mateus, J. et al. Selective and cross-reactive SARS-CoV-2 T cell epitopes in unexposed humans. Science 370, 89-94 (2020).
Grifoni, A. et al. Targets of T Cell Responses to SARS-CoV-2 Coronavirus in Humans with COVID-19 Disease and Unexposed Individuals. Cell 181, 1489-1501 e1415 (2020).
Le Bert, N. et al. SARS-CoV-2-specific T cell immunity in cases of COVID-19 and SARS, and
Braun, J. et al. SARS-CoV-2-reactive T cells in healthy donors and patients with COVID-19. Nature (2020).
Han, A., Glanville, J., Hansmann, L. & Davis, M. M. Linking T-cell receptor sequence to functional phenotype at the single-cell level. Nat Biotechnol 32, 684-692 (2014).
Huang, H., Wang, C., Rubelt, F., Scriba, T. J. & Davis, M. M. Analyzing the Mycobacterium tuberculosis immune response by T-cell receptor clustering with GLIPH2 and genome-wide antigen screening. Nat Biotechnol 38, 1194-1202 (2020).
Newell, E. W. & Davis, M. M. Beyond model antigens: high-dimensional methods for the analysis of antigen-specific T cells. Nat Biotechnol 32, 149-157 (2014).
Yang, J. et al. Searching immunodominant epitopes prior to epidemic: HLA class II-restricted SARS-CoV spike protein epitopes in unexposed individuals. International immunology 21, 63-71 (2009).
Saligrama, N. et al. Opposing T cell responses in experimental autoimmune encephalomyelitis. Nature 572, 481-487 (2019).
Sibener, L. V. et al. Isolation of a Structural Mechanism for Uncoupling T Cell Receptor Signaling from Peptide-MHC Binding. Cell 174, 672-687 e627 (2018).
Chen, B. et al. Predicting HLA class II antigen presentation through integrated deep learning. Nat Biotechnol 37, 1332-1343 (2019).
Nielsen, M., Andreatta, M., Peters, B. & Buus, S. Immunoinformatics: Predicting Peptide—MHC Binding. Annual Review of Biomedical Data Science 3, 191-215 (2020).
Reynisson, B. et al. Improved Prediction of MHC II Antigen Presentation through Integration and Motif Deconvolution of Mass Spectrometry MHC Eluted Ligand Data. J Proteome Res 19, 2304-2315 (2020).
Abelin, J. G. et al. Defining HLA-II Ligand Processing and Binding Rules with Mass Spectrometry Enhances Cancer Epitope Prediction. Immunity 51, 766-779 e717 (2019).
Nanaware, P. P., Jurewicz, M. M., Leszyk, J., Shaffer, S. A. & Stern, L. J. HLA-DO modulates the diversity of the MHC-II self-peptidome. Mol Cell Proteomics (2018).
Khodadoust, M. S. et al. Antigen presentation profiling reveals recognition of lymphoma immunoglobulin neoantigens. Nature 543, 723-727 (2017).
Purcell, A. W., Ramarathinam, S. H. & Ternette, N. Mass spectrometry-based identification of MHC-bound peptides for immunopeptidomics. Nat Protoc 14, 1687-1707 (2019).
Jiang, W. et al. pH-susceptibility of HLA-DO tunes DO/DM ratios to regulate HLA-DM catalytic activity. Scientific reports 5, 17333 (2015).
Sidney, J. et al. Divergent motifs but overlapping binding repertoires of six HLA-DQ molecules frequently expressed in the worldwide human population. J Immunol 185, 4189-4198 (2010).
Osterbye, T. et al. HLA Class II Specificity Assessed by High-Density Peptide Microarray Interactions. J Immunol 205, 290-299 (2020).
Justesen, S., Harndahl, M., Lamberth, K., Nielsen, L. L. & Buus, S. Functional recombinant MHC class II molecules and high-throughput peptide-binding assays. Immunome Res 5, 2 (2009).
Boder, E. T. & Wittrup, K. D. Yeast surface display for screening combinatorial polypeptide libraries. Nat Biotechnol 15, 553-557 (1997).
Boder, E. T. & Jiang, W. Engineering antibodies for cancer therapy. Annual review of chemical and biomolecular engineering 2, 53-75 (2011).
Busch, R., Pashine, A., Garcia, K. C. & Mellins, E. D. Stabilization of soluble, low-affinity HLA-DM/HLA-DR1 complexes by leucine zippers. J Immunol Methods 263, 111-121 (2002).
Serra, P. et al. Increased yields and biological potency of knob-into-hole-based soluble MHC class II molecules. Nature communications 10, 4917 (2019).
Jiang, W. & Boder, E. T. High-throughput engineering and analysis of peptide binding to class II MHC. Proc Natl Acad Sci USA 107, 13258-13263 (2010).
Boder, E. T., Bill, J. R., Nields, A. W., Marrack, P. C. & Kappler, J. W. Yeast surface display of a noncovalent MHC class II heterodimer complexed with antigenic peptide. Biotechnol Bioeng 92, 485-491 (2005).
Adler, L. N. et al. The Other Function: Class II-Restricted Antigen Presentation by B Cells. Frontiers in immunology 8, 319 (2017).
Mellins, E. D. & Stem, L. J. HLA-DM and HLA-DO, key regulators of MHC-II processing and presentation. Curr Opin Immunol 26, 115-122 (2014).
Rinderknecht, C. H. et al. Posttranslational Regulation of I-Ed by Affinity for CLIP. The Journal of Immunology 179, 5907 (2007).
Wen, F., Esteban, O. & Zhao, H. M. Rapid identification of CD4+ T-cell epitopes using yeast displaying pathogen-derived peptide library. Journal of Immunological Methods 336, 37-44 (2008).
Wen, F., Sethi, D. K., Wucherpfennig, K. W. & Zhao, H. Cell surface display of functional human MHC class II proteins: yeast display versus insect cell display. Protein Eng Des Sel 24, 701-709 (2011).
Birnbaum, M. E. et al. Deconstructing the peptide-MHC specificity of T cell recognition. Cell 157, 1073-1087 (2014).
Esteban, O. & Zhao, H. Directed evolution of soluble single-chain human class II MHC molecules. J Mol Biol 340, 81-95 (2004).
Han, F. et al. Narcolepsy onset is seasonal and increased following the 2009 H1N1 pandemic in China. Ann Neurol 70, 410-417 (2011).
Partinen, M. et al. Increased incidence and clinical picture of childhood narcolepsy following the 2009 H1N1 pandemic vaccination campaign in Finland. PLoS One 7, e33723 (2012).
Schinkelshoek, M. S. et al. H1N1 hemagglutinin-specific HLA-DQ6-restricted CD4+ T cells can be readily detected in narcolepsy type 1 patients and healthy controls. Journal of Neuroimmunology 332, 167-175 (2019).
Siebold, C. et al. Crystal structure of HLA-DQ0602 that protects against type 1 diabetes and confers strong susceptibility to narcolepsy. Proc Natl Acad Sci USA 101, 1999-2004 (2004).
Wrapp, D. et al. Cryo-EM structure of the 2019-nCoV spike in the prefusion conformation. Science 367, 1260-1263 (2020).
Cai, Y. et al. Distinct conformational states of SARS-CoV-2 spike protein. Science 369, 1586-1592 (2020).
Fast, E. & Chen, B. Potential T-cell and B-cell Epitopes of 2019-nCoV. bioRxiv (2020).
Grifoni, A. et al. A Sequence Homology and Bioinformatic Approach Can Predict Candidate Targets for Immune Responses to SARS-CoV-2. Cell host & microbe 27, 671-680.e672 (2020).
Starwalt, S. E., Masteller, E. L., Bluestone, J. A. & Kranz, D. M. Directed evolution of a single-chain class II MHC product by yeast display. Protein Eng 16, 147-156 (2003).
Feldhaus, M. J. et al. Flow-cytometric isolation of human antibodies from a nonimmune Saccharomyces cerevisiae surface display library. Nat Biotechnol 21, 163-170 (2003).
Korber, B. et al. Tracking Changes in SARS-CoV-2 Spike: Evidence that D614G Increases Infectivity of the COVID-19 Virus. Cell 182, 812-827.e819 (2020).
Hung, S. C. et al. Epitope Selection for HLA-DQ2 Presentation: Implications for Celiac Disease and Viral Defense. J Immunol 202, 2558-2569 (2019).

INFORMAL SEQUENCE LISTING

| SEQ ID NO | Name | Sequence |
|---|---|---|
| 1 | MHC-II HLA-DR4 alpha chain | IKEEHVIIQAEFYLNPDQSGEFMFDFDGDEIFHVDMAKKE |
| | ectodomain | TVWRLEEFGRFASFEAQGALANIAVDKANLEIMTKRSNYT |
| | | PITNVPPEVTVLTNSPVELREPNVLICFIDKFTPPVVNVT |
| | | WLRNGKPVTTGVSETVFLPREDHLFRKFHYLPFLPSTEDV |
| | | YDCRVEHWGLDEPLLKHWEFDAPSPLPETTEN |
| 2 | MHC-II HLA-DR4 beta chain | GDTRPRFLEQVKHECHFFNGTERVRFLDRYFYHQEEYVRF |
| | ectodomain | DSDVGEYRAVTELGRPDAEYWNSQKDLLEQKRAAVDTYCR |
| | | HNYGVGESFTVQRRVYPEVTVYPAKTQPLOHHNLLVCSVN |
| | | GFYPGSIEVRWFRNGQEEKTGVVSTGLIQNGDWTFQTLVM |
| | | LETVPRSGEVYTCQVEHPSLTSPLTVEWRARSESA |
| 3 | MHC-II HLA-DQ6 alpha chain | EDIVADHVASCGVNLYQFYGPSGQYTHEFDGDEQFYVDLE |
| | ectodomain | RKETAWRWPEFSKFGGFDPQGALRNMAVAKHNLNIMIKRY |
| | | NSTAATNEVPEVTVFSKSPVTLGQPNTLICLVDNIFPPVV |
| | | NITWLSNGQSVTEGVSETSFLSKSDHSFFKISYLTFLPSA |
| | | DEIYDCKVEHWGLDQPLLKHWEPEIPAPMSELTET |
| 4 | MHC-II HLA-DQ6 beta chain | PEDFVFQFKGMCYFTNGTERVRLVTRYIYNREEYARFDSD |
| | ectodomain | VGVYRAVTPQGRPDAEYWNSQKEVLEGTRAELDTVCRHNY |
| | | EVAFRGILQRRVEPTVTISPSRTEALNHHNLLVCSVTDFY |
| | | PGQIKVRWFRNDQEETAGVVSTPLIRNGDWTFQILVMLEM |
| | | TPQRGDVYTCHVEHPSLQSPITVEWRAQSESAQSK |
| 5 | Jun motif | RIARLEEKVKTLKAQNSELASTANMLREQVAQLKQKVMNH |
| 6 | Fos motif | LTDTLQAETDQLEDEKSALQTEIANLLKEKEKLEFILAAH |
| 7 | Clip Peptide | PVSKMRMATPLLMQA |
| 8 | c-Myc Tag | EQKLISEEDL |
| 9 | HA-Tag | YPYDVPDYA |
| 10 | DR4 alpha chain (MHC-II allele | MKVLIVLLAIFAALPLALAQPVISTTVGSAAEGSLDKREAR |
| | HLA-DRA*01:01) ORF protein | PIKEEHVIIQAEFYLNPDQSGEFMFDFDGDEIFHVDMAKKE |
| | construct | TVWRLEEFGRFASFEAQGALANIAVDKANLEIMTKRSNYTP |
| | (Start codon Syn-pre-pro leader | ITNVPPEVTVLTNSPVELREPNVLICFIDKFTPPVVNVTWL |
| | /DRA1*01:01/Jun motif/C- | RNGKPVTTGVSETVFLPREDHLFRKFHYLPFLPSTEDVYDC |
| | myc-tag/Stop codon) | RVEHWGLDEPLLKHWEFDAPSPLPETTENGAGGGGSLEVLF |
| | | QGPGGGRIARLEEKVKTLKAQNSELASTANMLREQVAQLKQ |
| | | KVMNHVDGGHHHHHHPWEQKLISEEDL |
| 11 | DR4 beta chain (MHC-II allele | MKVLIVLLAIFAALPLALAQPVISTTVGSAAEGSLDKREAR |
| | HLA-DRBl*04:01) ORF protein | GDTRPRFLEQVKHECHFFNGTERVRFLDRYFYHQEEYVRFD |
| | construct | SDVGEYRAVTELGRPDAEYWNSQKDLLEQKRAAVDTYCRHN |
| | (Start codon Syn-pre-pro leader | YGVGESFTVQRRVYPEVTVYPAKTQPLQHHNLLVCSVNGFY |
| | /DRB1*04:01/Fos motif/ | PGSIEVRWFRNGQEEKTGVVSTGLIQNGDWTFQTLVMLETV |
| | Aga2p/HA-tag/Stop codon) | PRSGEVYTCQVEHPSLTSPLTVEWRARSESALKGGGGSLEV |
| | | LFQGPGGGLTDTLQAETDQLEDEKSALQTEIANLLKEKEKL |
| | | EFILAAHTSGGDYKDDDDKGGGGSGGGGSQELTTICEQIPS |
| | | PTLESTPYSLSTTTILANGKAMQGVFEYYKSVTFVSNCGSH |
| | | PSTTSKGSPINTQYVFKDNSSTIEGRYPYDVPDYA |
| 12 | DQ6 alpha chain (MHC-IIallele | MKVLIVLLAIFAALPLALAQPVISTTVGSAAEGSLDKREAR |
| | HLA-DQA1*01:02) ORF protein | EDIVADHVASCGVNLYQFYGPSGQYTHEFDGDEQFYVDLER |
| | construct | KETAWRWPEFSKFGGFDPQGALRNMAVAKHNLNIMIKRYNS |
| | (Start codon Syn-pre-pro leader | TAATNEVPEVTVFSKSPVTLGQPNTLICLVDNIFPPVVNIT |
| | /DQA1*01:02/Fos motif/ | WLSNGQSVTEGVSETSFLSKSDHSFFKISYLTFLPSADEIY |
| | Aga2p/HA-tag/Stop codon) | DCKVEHWGLDQPLLKHWEPEIPAPMSELTETGGGGSLEVLF |
| | | QGPGGGLTDTLQAETDQLEDEKSALQTEIANLLKEKEKLEF |
| | | ILAAHTSGGDYKDDDDKGGGGSGGGGSQELTTICEQIPSPT |
| | | LESTPYSLSTTTILANGKAMQGVFEYYKSVTFVSNCGSHPS |
| | | TTSKGSPINTQYVFKDNSSTIEGRYPYDVPDYA |
| 13 | DQ6 beta chain (MHC-II allele | MKVLIVLLAIFAALPLALAQPVISTTVGSAAEGSLDKREAR |
| | HLA-DQB1* 06:02) ORF protein | PPEDFVFQFKGMCYFTNGTERVRLVTRYIYNREEYARFDSD |
| | construct | VGVYRAVTPQGRPDAEYWNSQKEVLEGTRAELDTVCRHNYE |
| | (Start codon Syn-pre-pro leader | VAFRGILQRRVEPTVTISPSRTEALNHHNLLVCSVTDFYPG |
| | /DQB1*06:02/Jun motif/C- | QIKVRWFRNDQEETAGVVSTPLIRNGDWTFQILVMLEMTPQ |
| | myc-tag/Stop codon) | RGDVYTCHVEHPSLQSPITVEWRAQSESAQSKGTGGGGSLE |
| | | VLFQGPGGGRIARLEEKVKTLKAQNSELASTANMLREQVAQ |
| | | LKQKVMNHVDGGHHHHHHPWEQKLISEEDL |
| 14 | DQ6 alpha chain (MHC-II allele | MKVLIVLLAIFAALPLALAQPVISTTVGSAAEGSLDKREAR |
| | HLA-DQAl*01:02) ORF protein | EDIVADHVASCGVNLYQFYGPSGQYTHEFDGDEQFYVDLER |
| | construct | KETAWRWPEFSKFGGFDPQGALRNMAVAKHNLNIMIKRYNS |
| | (Start codon Syn-pre-pro leader | TAATNEVPEVTVFSKSPVTLGQPNTLICLVDNIFPPVVNIT |
| | /DQA1*01:02/Fos motif/ | WLSNGQSVTEGVSETSFLSKSDHSFFKISYLTFLPSADEIY |
| | Aga2p/HA-tag/Stop codon) | DCKVEHWGLDQPLLKHWEPEIPAPMSELTETGGGGSLEVLF |
| | | QGPGGGLTDTLQAETDQLEDEKSALQTEIANLLKEKEKLEF |
| | | ILAAHTSGGDYKDDDDKGGGGSGGGGSQELTTICEQIPSPT |
| | | LESTPYSLSTTTILANGKAMQGVFEYYKSVTFVSNCGSHPS |
| | | TTSKGSPINTQYVFKDNSSTIEGRYPYDVPDYA |
| 15 | DQ6 beta chain (MHC-II allele | MKVLIVLLAIFAALPLALAQPVISTTVGSAAEGSLDKREAR |
| | HLA-DQB1* 06:02) ORF protein | PPVSKMRMATPLLMQAGGGGSLVPRGSGGGGSPEDFVFQF |
| | construct | KGMCYFTNGTERVRLVTRYIYNREEYARFDSDVGVYRAVT |
| | (Start codon Syn-pre-pro leader | PQGRPDAEYWNSQKEVLEGTRAELDTVCRHNYEVAFRGIL |
| | /CLIP/DQB1*06:02/Jun | QRRVEPTVTISPSRTEALNHHNLLVCSVTDFYPGQIKVRW |
| | motif/C-myc-tag/Stop | FRNDQEETAGVVSTPLIRNGDWTFQILVMLEMTPQRGDVY |
| | codon) | TCHVEHPSLQSPITVEWRAQSESAQSKGTGGGGSLEVLFQ |
| | | GPGGGRIARLEEKVKTLKAQNSELASTANMLREQVAQLKQ |
| | | KVMNHVDGGHHHHHHPWEQKLISEEDL |
| 16 | Aga2p | QELTTICEQIPSPTLESTPYSLSTTTILANGKAMQGVFEY |
| | | YKSVTFVSNCGSHPSTTSKGSPINTQYVFKDNSSTIEGR |
| 17 | Syn-pre-pro leader | KVLIVLLAIFAALPLALAQPVISTTVGSAAEGSLDKREAR |
| | | P |
| 18 | Peptide linker | GAGGGGSLEVLFQGPGGG |
| 19 | Peptide linker | VDGGHHHHHHPW |
| | (His Tag in Bold) | |
| 20 | Peptide linker | LKGGGGSLEVLFQGPGGG |
| 21 | Peptide linker | TSGGDYKDDDDKGGGGSGGGGS |
| | (Flag-tag in bold) | |
| 22 | Peptide linker | GGGGSLEVLFQGPGGG |
| 23 | Peptide linker | GTGGGGSLEVLFQGPGGG |

What is claimed is:

1. A cell comprising a major histocompatibility class II (MHC II) complex, wherein
said MHC II complex comprises an alpha chain and a beta chain, wherein
said alpha chain is attached to a first protein binding domain and said beta chain is attached to a second protein binding domain, wherein
said first protein binding domain is bound to said second protein binding domain to form a MHC II complex.

2. The cell of claim 1, wherein said cell is a yeast cell.

3. The cell of claim 1, wherein said MHC II complex is bound to the surface of said cell through attachment of said alpha chain or said beta chain to a molecule on said cell surface.

4. The cell of claim 3, wherein said alpha chain or said beta chain is covalently attached to said molecule on said cell surface.

5. The cell of claim 3, wherein said molecule is a protein.

6. The cell of claim 5, wherein said protein is endogenous to said cell.

7. The cell of claim 5, wherein said protein is Aga2p, a-agglutinin, α-agglutinin, flocculin, Cwp1p, Cwp2p or Tip1p.

8. The cell of claim 5, wherein said protein is Aga2p.

9. The cell of claim 8, wherein said Aga2p has the amino acid sequence of SEQ ID NO:16.

10. The cell of claim 1, wherein said first protein binding domain is non-covalently bound to said second protein binding domain.

11. The cell of claim 1, wherein said first protein binding domain is covalently bound to said second protein.

12. The cell of claim 1, wherein said first protein binding domain is a first leucine zipper domain and said second protein binding domain is a second leucine zipper domain.

13. The cell of claim 12, wherein said first leucine zipper domain has the sequence of SEQ ID NO:5 and said second leucine zipper domain has the sequence of SEQ ID NO:6.

14. The cell of claim 12, wherein said first leucine zipper domain has the sequence of SEQ ID NO:6 and said second leucine zipper domain has the sequence of SEQ ID NO:5.

15. The cell of claim 1, wherein said first protein binding domain is attached to the C-terminus of said alpha chain and said second protein binding domain is attached to the C-terminus of said beta chain.

16. The cell of claim 1, wherein said alpha chain is an HLA-DR4 alpha chain and said beta chain is an HLA-DR4 beta chain.

17. The cell of claim 16, wherein said HLA-DR4 alpha chain comprises the amino acid sequence of SEQ ID NO:1.

18. The cell of claim 16, wherein said HLA-DR4 beta chain comprises the amino acid sequence of SEQ ID NO:2.

19. The cell of claim 1, wherein said alpha chain is an HLA-DQ6 alpha chain and said beta chain is an HLA-DQ6 beta chain.

20. The cell of claim 19, wherein said HLA-DQ6 alpha chain comprises the amino acid sequence of SEQ ID NO:3.

21. The cell of claim 19, wherein said HLA-DQ6 beta chain comprises the amino acid sequence of SEQ ID NO:4.

22. A nucleic acid encoding a major histocompatibility class II (MHC II) complex, wherein
said MHC II complex comprises an alpha chain and a beta chain, wherein
said alpha chain is attached to a first protein binding domain and said beta chain is attached to a second protein binding domain, wherein
said first protein binding domain and said second protein binding domain are capable of non-covalently binding to form a MHC complex.

23. A method of making a major histocompatibility class II (MHC II) complex, the method comprising transforming a cell with the nucleic acid of claim 22, and culturing said cell under conditions wherein said MHC II complex is expressed.

24. The method of claim 23, wherein said cell is a yeast cell.

25. A method of identifying a peptide that binds a major histocompatibility class II (MHC II) complex, the method comprising:
i) contacting the cell of claim 1 with a peptide, and
ii) detecting binding of said peptide to said MHC II complex, thereby identifying said MHC II complex binding peptide.

26. The method of claim 25, wherein said peptide comprises a mixture of peptides

27. The method of claim 25, wherein said peptide competes with a reference peptide for binding of said MHC II complex.
